(19) United States
(12) Patent Application Publication
(10) Pub. No.: US 2024/0182921 A1
(43) Pub. Date: Jun. 6, 2024

(54) COMPOSITIONS AND METHODS FOR THE TREATMENT OF HEMOPHILIA

(71) Applicant: Poseida Therapeutics, Inc., San Diego, CA (US)

(72) Inventors: Julian David DOWN, San Diego, CA (US); Xinggang LIU, San Diego, CA (US); Jingjing JIANG, San Diego, CA (US)

(21) Appl. No.: 18/279,434

(22) PCT Filed: Mar. 4, 2022
(86) PCT No.: PCT/US2022/018976

§ 371 Date: Aug. 30, 2023

Related U.S. Application Data
(60) Provisional application No. 63/156,657, filed on Mar. 4, 2021.

(51) Int. Cl. C12N 15/86; A61K 48/00; A61P 7/04

(52) U.S. Cl. CPC C12N 15/86; A61K 48/0066; A61P 7/04; C12N 2750/14143; C12N 2800/90

(57) ABSTRACT

The present disclosure is directed to compositions and methods for the treatment of hemophilia. The compositions and methods can comprise an adeno-associated virus (AAV) piggyBac polynucleotide comprising a transgene. The transgene may comprise ornithine Factor VIII (FVIII) or Factor IX (FIX).

### RELATED APPLICATIONS

This application claims priority to, and the benefit of, U.S. Provisional Application No. 63/156,657, filed on Mar. 4, 2021, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Mar. 4, 2022, is named “POTH-068_001WO_SeqList.txt” and is about 277,932 bytes in size.

### BACKGROUND

Hemophilia is an inherited genetic disorder that impairs blood clotting in a subject. This impairment in blood clotting means that subjects with hemophilia bleed for longer time periods following an injury, more easily bruise, and suffer from an increased risk of bleeding inside the joints or in the brain. Hemophilia is most typically inherited from a subject's parents through an X chromosome which carries a nonfunctional gene.

The two most prevalent forms of Hemophilia are Hemophilia A and Hemophilia B. Hemophilia A is characterized by reduced levels of clotting Factor VIII (FVIII) and Hemophilia B is characterized by reduced levels of clotting Factor IX (FIX). Other forms of Hemophilia include Hemophilia C, which is characterized by reduced levels of Factor XI (FXI), and Parahemophilia, which is characterized by reduced levels of Factor V (FV).

Currently there are no cures for hemophilia, and treatment is limited to the administration of clotting factors. Thus, there is a long-felt need in the art for compositions and methods directed to the treatment of hemophilia. The present disclosure addresses this need and provides compositions and methods for gene therapy for Hemophilia, more specifically, Hemophilia A and Hemophilia B.

Previous attempts at developing gene therapies for the treatment of hemophilia, have suffered from the inability to produce long-term expression of the delivered transgene in the target tissues. The compositions and methods of the present disclosure provide a solution to this long felt-need in the art by providing transposon/transposase-based AAV vectors that yield long-term expression of the delivered transgene in targeted tissues.

### SUMMARY

The present disclosure relates to compositions and methods for the treatment of hemophilia in a subject in need thereof.

The present disclosure provides compositions comprising adeno-associated virus (AAV) piggyBac transposon polynucleotides.

In some aspects an AAV piggyBac transposon polynucleotide can comprise at least one AAV inverted terminal repeat (ITR) sequence. In some aspects, an AAV piggyBac transposon polynucleotide can comprise at least one piggyBac ITR sequence. In some aspects, an AAV piggyBac transposon polynucleotide can comprise at least one insulator sequence. In some aspects an AAV piggyBac transposon polynucleotide can comprise at least one promoter sequence. In some aspects, an AAV piggyBac transposon polynucleotide can comprise at least one transgene sequence. In some aspects, an AAV piggyBac transposon polynucleotide can comprise at least one polyA sequence. In some aspects, an AAV piggyBac transposon polynucleotide can comprise at least one self-cleaving peptide sequence. In some aspects, an AAV piggyBac transposon polynucleotide can comprise at least one DNA spacer sequence. In some aspects, an AAV piggyBac transposon polynucleotide can comprise at least one nucleic acid sequence.

An AAV piggyBac transposon polynucleotide can comprise a first AAV ITR sequence, a first piggyBac ITR sequence, a first insulator sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, a second insulator sequence, a second piggyBac ITR sequence, and a second AAV ITR sequence.

The present disclosure provides AAV vectors comprising the AAV piggyBac transposon polynucleotides of the present disclosure.

The present disclosure provides methods of treating hemophilia in a subject comprising administering the AAV vectors of the present disclosure to the subject.

Any of the aspects and/or embodiments described herein can be combined with any other aspect and/or embodiment described herein.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a schematic of an exemplary AAV piggyBac transposon polynucleotide of the present disclosure.

FIG. 2A is a schematic of an exemplary AAV piggyBac transposon polynucleotide of the present disclosure.

FIG. 2B is a schematic of an exemplary AAV piggyBac transposon polynucleotide of the present disclosure.

FIG. 2C is a schematic of an exemplary AAV piggyBac transposon polynucleotide of the present disclosure.

FIG. 3A is a schematic of an exemplary AAV transposase polynucleotide of the present disclosure.

FIG. 3B is a schematic of an exemplary AAV transposase polynucleotide of the present disclosure.

FIG. 4 is a schematic of an exemplary AAV piggyBac transposon polynucleotide of the present disclosure.

FIG. 5 is a graph showing the levels of human Factor IX protein in mice administered various vectors of the present disclosure.

FIG. 6 is a graph showing the levels of human Factor IX protein in mice administered various vectors of the present disclosure.

### DETAILED DESCRIPTION

The present disclosure provides for compositions and methods for the treatment of hemophilia. The compositions and methods are described in further detail herein.

### Compositions of Disclosure

Adeno-Associated Virus (AAV) piggyBac Transposon Polynucleotides

The present disclosure provides compositions comprising adeno-associated virus (AAV) piggyBac transposon polynucleotides.

In some aspects an AAV piggyBac transposon polynucleotide can comprise at least one AAV inverted terminal repeat (ITR) sequence. In some aspects, an AAV piggyBac transposon polynucleotide can comprise at least one piggyBac ITR sequence. In some aspects, an AAV piggyBac transposon polynucleotide can comprise at least one insulator sequence. In some aspects an AAV piggyBac transposon polynucleotide can comprise at least one promoter sequence. In some aspects, an AAV piggyBac transposon polynucleotide can comprise at least one transgene sequence. In some aspects, an AAV piggyBac transposon polynucleotide can comprise at least one polyA sequence. In some aspects, an AAV piggyBac transposon polynucleotide can comprise at least one self-cleaving peptide sequence. In some aspects, an AAV piggyBac transposon polynucleotide can comprise at least one DNA spacer sequence. In some aspects, an AAV piggyBac transposon polynucleotide can comprise at least one nucleic acid sequence.

An AAV piggyBac transposon polynucleotide can comprise a first AAV ITR sequence, a first piggyBac ITR sequence, a first insulator sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by a second insulator sequence, followed by a second piggyBac ITR sequence, and followed by a second AAV ITR sequence.

An AAV piggyBac transposon polynucleotide can comprise a first AAV ITR sequence, a first piggyBac ITR sequence, a first insulator sequence, a second insulator sequence, a second piggyBac ITR sequence, and a second AAV ITR sequence, wherein between the first insulator sequence and the second insulator sequence there is any combination of at least one promoter sequence, at least one transgene sequence, at least one self-cleaving peptide sequence, and at least one polyA sequence.

In some aspects, an AAV piggyBac transposon polynucleotide can comprise in the 5′ to 3′ direction a first AAV ITR sequence, a first piggyBac ITR sequence, a first insulator sequence, a second insulator sequence, a second piggyBac ITR sequence, and a second AAV ITR sequence, wherein between the first insulator sequence and the second insulator sequence there is any combination of at least one promoter sequence, at least one transgene sequence, at least one self-cleaving peptide sequence, and at least one polyA sequence.

In some aspects, an AAV piggyBac transposon polynucleotide can comprise a first AAV ITR sequence, followed by a first piggyBac ITR sequence, followed by a first insulator sequence, followed by a second insulator sequence, followed by a second piggyBac ITR sequence, and followed by a second AAV ITR sequence, wherein between the first insulator sequence and the second insulator sequence there is any combination of at least one promoter sequence, at least one transgene sequence, at least one self-cleaving peptide sequence, and at least one polyA sequence.

An AAV piggyBac transposon polynucleotide can comprise a first AAV ITR sequence, a first piggyBac ITR sequence, a first insulator sequence, at least one promoter sequence, at least one transgene sequence, a polyA sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, at least one transgene sequence, a polyA sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, and followed by a second AAV ITR sequence.

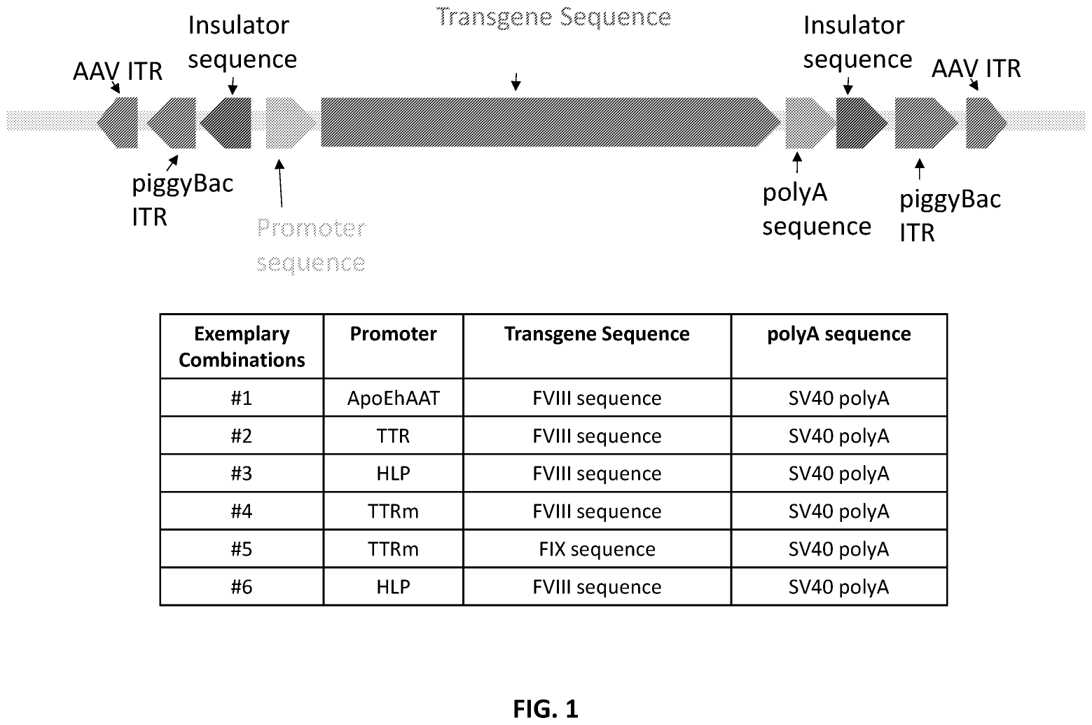

A schematic of the preceding piggyBac transposon polynucleotide is shown in FIG. 1.

In a non-limiting example of the preceding AAV piggyBac transposon polynucleotides, the at least one promoter sequence can comprise an ApoEhAAT promoter sequence and the at least one transgene sequence can comprise a nucleic acid sequence that encodes for a Factor VIII (FVIII) polypeptide. This non-limiting example of an AAV piggyBac transposon polynucleotide is shown in FIG. 1.

In a non-limiting example of the preceding AAV piggyBac transposon polynucleotides, the at least one promoter sequence can comprise an TTR promoter sequence and the at least one transgene sequence can comprise a nucleic acid sequence that encodes for a FVIII polypeptide. This non-limiting example of an AAV piggyBac transposon polynucleotide is shown in FIG. 1.

In a non-limiting example of the preceding AAV piggyBac transposon polynucleotides, the at least one promoter sequence can comprise an HLP promoter sequence and the at least one transgene sequence can comprise a nucleic acid sequence that encodes for a FVIII polypeptide. This non-limiting example of an AAV piggyBac transposon polynucleotide is shown in FIG. 1.

In a non-limiting example of the preceding AAV piggyBac transposon polynucleotides, the at least one promoter sequence can comprise an TTRm promoter sequence and the at least one transgene sequence can comprise a nucleic acid sequence that encodes for a FVIII polypeptide. This non-limiting example of an AAV piggyBac transposon polynucleotide is shown in FIG. 1.

In a non-limiting example of the preceding AAV piggyBac transposon polynucleotides, the at least one promoter sequence can comprise an TTRm promoter sequence and the at least one transgene sequence can comprise a nucleic acid sequence that encodes for a Factor IX (FIX) polypeptide. This non-limiting example of an AAV piggyBac transposon polynucleotide is shown in FIG. 1.

In a non-limiting example of the preceding AAV piggyBac transposon polynucleotides, the at least one promoter sequence can comprise an HLP promoter sequence and the at least one transgene sequence can comprise a nucleic acid sequence that encodes for a FIX polypeptide. This non-limiting example of an AAV piggyBac transposon polynucleotide is shown in FIG. 1.

An AAV piggyBac transposon polynucleotide can comprise a first AAV ITR sequence, a first piggyBac ITR sequence, a first insulator sequence, at least one promoter sequence, at least one transgene sequence, a polyA sequence, a second insulator sequence, a second piggyBac ITR sequence, at least one DNA spacer sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, at least one transgene sequence, a polyA sequence, a second insulator sequence, a second piggyBac ITR sequence, at least one DNA spacer sequence and a second AAV ITR sequence.

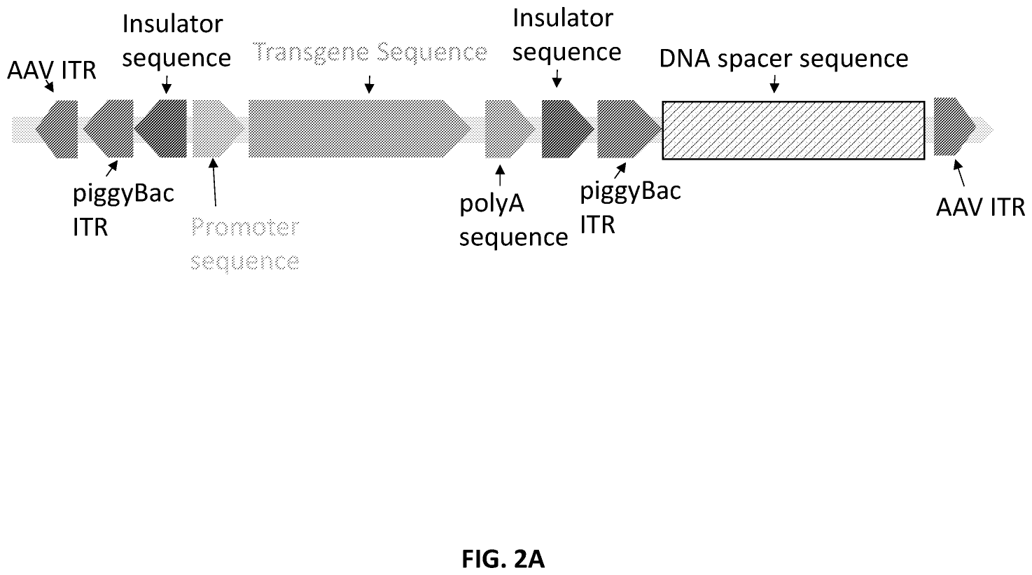

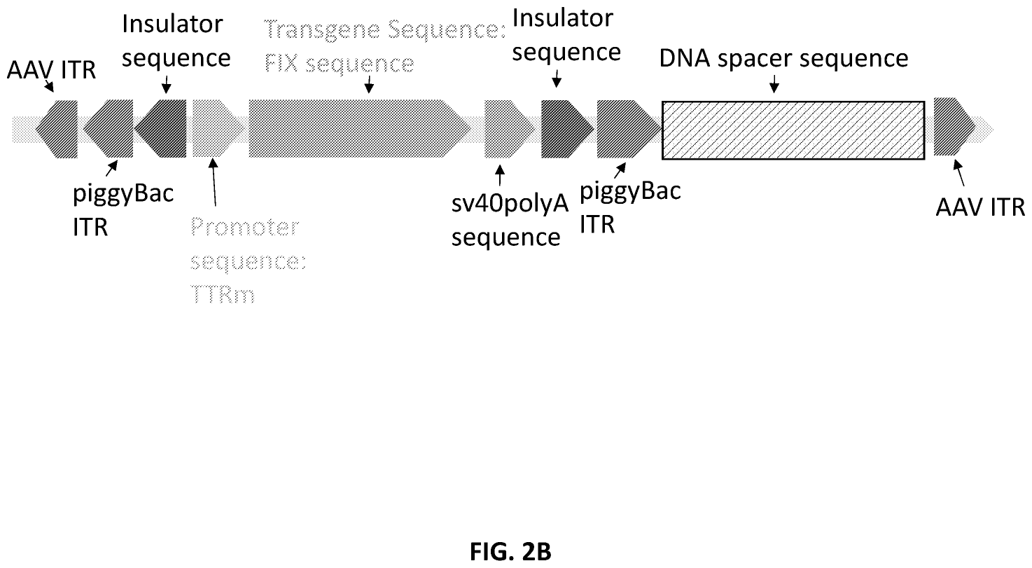

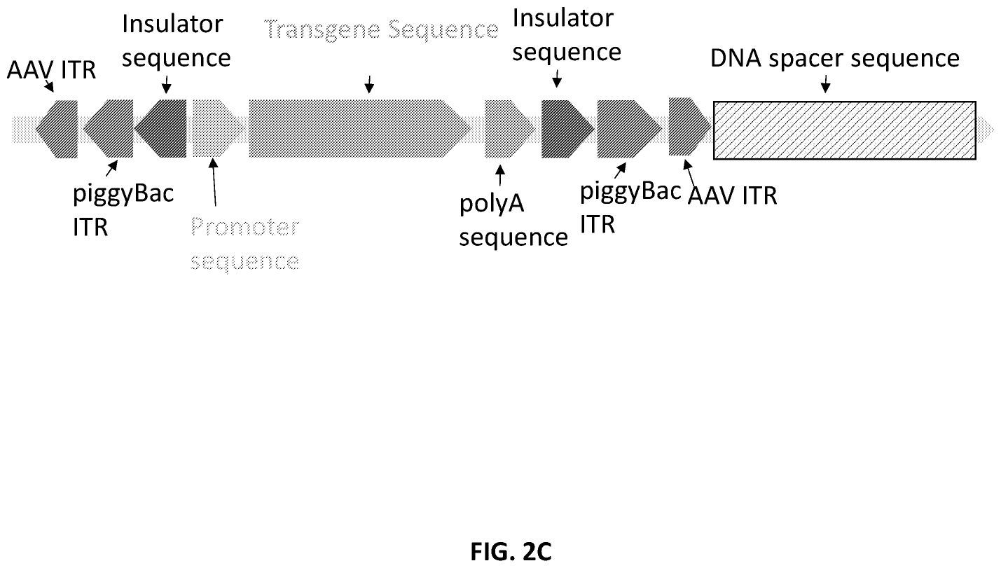

In some aspects, an AAV piggyBac transposon polynucleotide can comprise a first AAV ITR sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, followed by at least one DNA spacer sequence and followed by a second AAV ITR sequence. A schematic of the preceding AAV piggyBac transposon polynucleotide is shown in FIG. 2A

In a non-limiting example of the preceding AAV piggyBac transposon polynucleotides, the at least one promoter sequence can comprise a TTRm promoter sequence and the at least one transgene sequence can comprise a nucleic acid sequence that encodes for a FIX polypeptide. This non-limiting example of an AAV piggyBac transposon polynucleotide is shown in FIG. 2B.

In some aspects, an AAV piggyBac transposon polynucleotide can comprise in between a second piggyBac ITR sequence and a second AAV ITR sequence, at least one DNA spacer sequence.

An AAV piggyBac transposon polynucleotide can comprise a first AAV ITR sequence, a first piggyBac ITR sequence, a first insulator sequence, at least one promoter sequence, at least one transgene sequence, a polyA sequence, a second insulator sequence, a second piggyBac ITR sequence, a second AAV ITR sequence and at least one DNA spacer sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, at least one transgene sequence, a polyA sequence, a second insulator sequence, a second piggyBac ITR sequence, a second AAV ITR sequence and at least one DNA spacer sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, followed by a second AAV ITR sequence and followed by at least one DNA spacer sequence.

A schematic of the preceding AAV piggyBac transposon polynucleotide is shown in FIG. 2C.

An AAV piggyBac transposon polynucleotide can comprise a first AAV ITR sequence, a first piggyBac ITR sequence, a first insulator sequence, at least one promoter sequence, a first transgene sequence, at least one self-cleaving peptide sequence, an at least second transgene sequence, a polyA sequence, a second insulator sequence, a second piggyBac ITR sequence, and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, a first transgene sequence, at least one self-cleaving peptide sequence, an at least second transgene sequence, a polyA sequence, a second insulator sequence, a second piggyBac ITR sequence, and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by a first transgene sequence, followed by at least one self-cleaving peptide sequence, followed by an at least second transgene sequence, followed by a polyA sequence, followed by a second insulator sequence, followed by a second piggyBac ITR sequence, and followed by a second AAV ITR sequence.

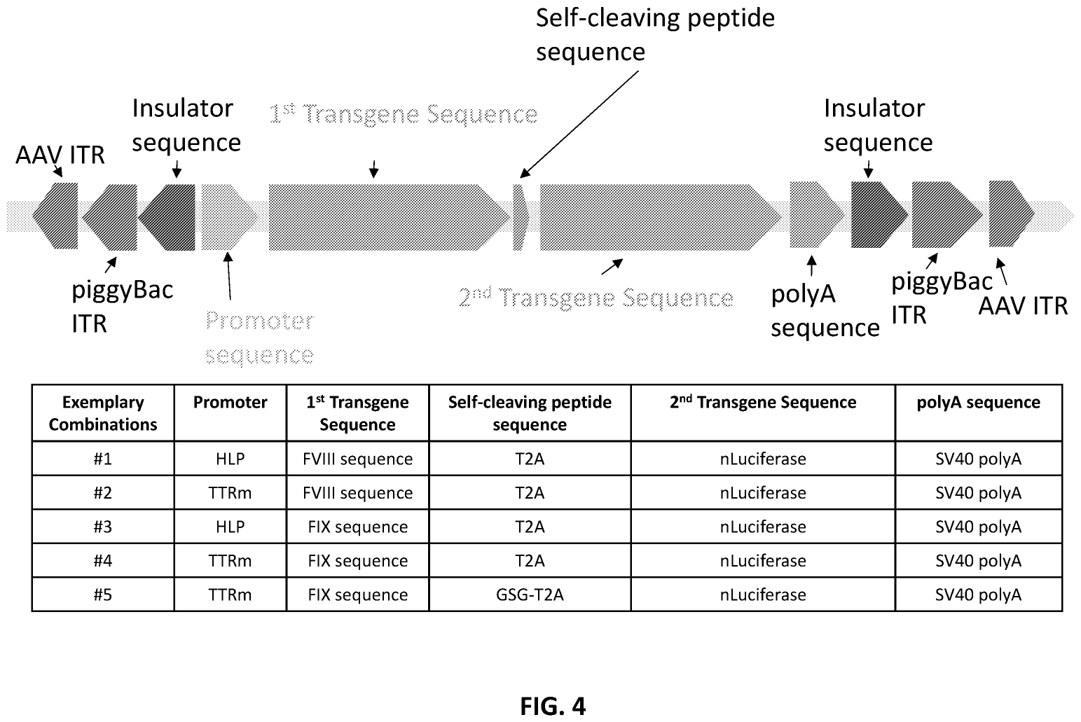

A schematic of the preceding AAV piggyBac transposon polynucleotide is shown in FIG. 4.

In another non-limiting example of the preceding AAV piggyBac transposon polynucleotides the at least one promoter sequence can comprise an HLP promoter sequence, the first transgene sequence can comprise a nucleic acid sequence that encodes for FVIII polypeptide, the at least one self-cleaving peptide sequence can comprise that encodes for a T2A peptide and the at least second transgene sequence can comprise a luciferase sequence (e.g. NanoLuc). This non-limiting example of an AAV piggyBac transposon polynucleotide is shown in FIG. 4.

In another non-limiting example of the preceding AAV piggyBac transposon polynucleotides the at least one promoter sequence can comprise an TTRm promoter sequence, the first transgene sequence can comprise a nucleic acid sequence that encodes for FVIII polypeptide, the at least one self-cleaving peptide sequence can comprise that encodes for a T2A peptide and the at least second transgene sequence can comprise a luciferase sequence (e.g. NanoLuc). This non-limiting example of an AAV piggyBac transposon polynucleotide is shown in FIG. 4.

In another non-limiting example of the preceding AAV piggyBac transposon polynucleotides the at least one promoter sequence can comprise an HLP promoter sequence, the first transgene sequence can comprise a nucleic acid sequence that encodes for FIX polypeptide, the at least one self-cleaving peptide sequence can comprise that encodes for a T2A peptide and the at least second transgene sequence can comprise a luciferase sequence (e.g. NanoLuc). This non-limiting example of an AAV piggyBac transposon polynucleotide is shown in FIG. 4.

In another non-limiting example of the preceding AAV piggyBac transposon polynucleotides the at least one promoter sequence can comprise an TTRm promoter sequence, the first transgene sequence can comprise a nucleic acid sequence that encodes for FIX polypeptide, the at least one self-cleaving peptide sequence can comprise that encodes for a T2A peptide and the at least second transgene sequence can comprise a luciferase sequence (e.g. NanoLuc). This non-limiting example of an AAV piggyBac transposon polynucleotide is shown in FIG. 4.

In another non-limiting example of the preceding AAV piggyBac transposon polynucleotides the at least one promoter sequence can comprise an TTRm promoter sequence, the first transgene sequence can comprise a nucleic acid sequence that encodes for FIX polypeptide, the at least one self-cleaving peptide sequence can comprise that encodes for a GSG-T2A peptide and the at least second transgene sequence can comprise a luciferase sequence (e.g. NanoLuc). This non-limiting example of an AAV piggyBac transposon polynucleotide is shown in FIG. 4.

An AAV piggyBac transposon polynucleotide can comprise a first AAV ITR sequence, a first piggyBac ITR sequence, a first insulator sequence, a first promoter sequence, a first transgene sequence, at least a second promoter sequence, an at least second transgene sequence, a polyA sequence, a second insulator sequence, a second piggyBac ITR sequence, and a second AAV ITR sequence.

An AAV piggyBac transposon polynucleotide can comprise in the 5′ to 3′ direction a first AAV ITR sequence, a first piggyBac ITR sequence, a first insulator sequence, a first promoter sequence, a first transgene sequence, an at least a second promoter sequence, an at least second transgene sequence, a polyA sequence, a second insulator sequence, a second piggyBac ITR sequence, and a second AAV ITR sequence.

An AAV piggyBac transposon polynucleotide can comprise a first AAV ITR sequence, followed by a first piggyBac ITR sequence, followed by a first insulator sequence, followed by a first promoter sequence, followed by a first transgene sequence, followed by an at least a second promoter sequence, followed by an at least second transgene sequence, followed by a polyA sequence, followed by a second insulator sequence, followed by a second piggyBac ITR sequence and followed by a second AAV ITR sequence.

In some aspects, an AAV piggyBac transposon polynucleotide can comprise more than one transgene sequence. In some aspects wherein the AAV piggyBac transposon polynucleotide comprises more than one transgene sequence, individual transgene sequences can be separated by a self-cleaving peptide sequence. In some aspects wherein the AAV piggyBac transposon polynucleotide comprises more than one self-cleaving peptide sequence, the self-cleaving peptide sequences can be the same or can be different.

In some aspects, an AAV piggyBac transposon polynucleotide can comprise more than one transgene sequence. In some aspects wherein the AAV piggyBac transposon polynucleotide comprises more than one transgene sequence, the AAV piggyBac transposon may comprise multiple copies of a nucleic acid sequence that encodes for the same polypeptide.

In some aspects, an AAV piggyBac transposon polynucleotide can comprise more than one promoter sequence. In some aspects wherein the AAV piggyBac transposon polynucleotide comprises more than one promoter sequence, the promoter sequences can be the same or the promoter sequences can be different.

In some aspects, an AAV piggyBac transposon polynucleotide can comprise a first AAV ITR sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by a first transgene sequence, followed by a first self-cleaving peptide sequence, followed by a second transgene sequence, followed by an at least second self-cleaving peptide sequence, followed by at least a third transgene sequence, followed by a polyA sequence, followed by a second insulator sequence and followed by a second AAV ITR sequence.

In some aspects, an AAV piggyBac transposon polynucleotide can comprise a first AAV ITR sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence, a second piggyBac ITR sequence and a second AAV ITR sequence.

In some aspects, an AAV piggyBac transposon polynucleotide can comprise in the 5′ to 3′ direction a first AAV ITR sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence, a second piggyBac ITR sequence and a second AAV ITR sequence.

In some aspects, an AAV piggyBac transposon polynucleotide can comprise a first AAV ITR sequence, followed by a first piggyBac ITR sequence, followed by a first insulator sequence, followed by at least one promoter sequence, followed by a first transgene sequence, followed by a first self-cleaving peptide sequence, followed by a second transgene sequence, followed by an at least second self-cleaving peptide sequence, followed by at least a third transgene sequence, followed by a polyA sequence, followed by a second insulator sequence, followed by a second piggyBac ITR sequence and followed by a second AAV ITR sequence

An AAV piggyBac transposon polynucleotide can comprise a first nucleic acid sequence, a first piggyBac ITR sequence, a first insulator sequence, a second insulator sequence, a second piggyBac ITR sequence, and a second nucleic acid sequence.

In some aspects, an AAV piggyBac transposon polynucleotide can comprise in the 5′ to 3′ direction a first nucleic acid sequence, a first piggyBac ITR sequence, a first insulator sequence, a second insulator sequence, a second piggyBac ITR sequence, and a second nucleic acid sequence.

In some aspects, an AAV piggyBac transposon polynucleotide can comprise a first nucleic acid sequence, followed by a first piggyBac ITR sequence, followed by a first insulator sequence, followed by a second insulator sequence, followed by a second piggyBac ITR sequence, and followed by a second nucleic acid sequence.

An AAV piggyBac transposon polynucleotide can comprise a first nucleic acid sequence, a first piggyBac ITR sequence, a first insulator sequence, a second insulator sequence, a second piggyBac ITR sequence, and a second nucleic acid sequence, wherein between the first insulator sequence and the second insulator sequence there is any combination of at least one promoter sequence, at least one transgene sequence, at least one self-cleaving peptide sequence, and at least one polyA sequence.

In some aspects, an AAV piggyBac transposon polynucleotide can comprise in the 5′ to 3′ direction a first nucleic acid sequence, a first piggyBac ITR sequence, a first insulator sequence, a second insulator sequence, a second piggyBac ITR sequence, and a second nucleic acid sequence, wherein between the first insulator sequence and the second insulator sequence there is any combination of at least one promoter sequence, at least one transgene sequence, at least one self-cleaving peptide sequence, and at least one polyA sequence.

In some aspects, an AAV piggyBac transposon polynucleotide can comprise a first nucleic acid sequence, followed by a first piggyBac ITR sequence, followed by a first insulator sequence, followed by a second insulator sequence, followed by a second piggyBac ITR sequence, and followed by a second nucleic acid sequence, wherein between the first insulator sequence and the second insulator sequence there is any combination of at least one promoter sequence, at least one transgene sequence, at least one self-cleaving peptide sequence, and at least one polyA sequence.

An AAV piggyBac transposon polynucleotide can comprise a first nucleic acid sequence, a first piggyBac ITR sequence, a first insulator sequence, at least one promoter sequence, at least one transgene sequence, a polyA sequence, a second insulator sequence, a second piggyBac ITR sequence, and a second nucleic acid sequence.

In some aspects, an AAV piggyBac transposon polynucleotide can comprise in the 5′ to 3′ direction a first nucleic acid sequence, a first piggyBac ITR sequence, a first insulator sequence, at least one promoter sequence, at least one transgene sequence, a polyA sequence, a second insulator sequence, a second piggyBac ITR sequence, and a second nucleic acid sequence.

In some aspects, an AAV piggyBac transposon polynucleotide can comprise a first nucleic acid sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, and followed by a second nucleic acid sequence.

An AAV piggyBac transposon polynucleotide can comprise a first nucleic acid sequence, a first piggyBac ITR sequence, a first insulator sequence, at least one promoter sequence, at least one transgene sequence, a polyA sequence, a second insulator sequence, a second piggyBac ITR sequence, at least one DNA spacer sequence and a second nucleic acid sequence.

In some aspects, an AAV piggyBac transposon polynucleotide can comprise in the 5′ to 3′ direction a first nucleic acid sequence, a first piggyBac ITR sequence, a first insulator sequence, at least one promoter sequence, at least one transgene sequence, a polyA sequence, a second insulator sequence, a second piggyBac ITR sequence, at least one DNA spacer sequence and a second nucleic acid sequence.

In some aspects, an AAV piggyBac transposon polynucleotide can comprise a first nucleic acid sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, followed by at least one DNA spacer sequence and followed by a second nucleic acid sequence.

An AAV piggyBac transposon polynucleotide can comprise a first nucleic acid sequence, a first piggyBac ITR sequence, a first insulator sequence, at least one promoter sequence, at least one transgene sequence, a polyA sequence, a second insulator sequence, a second piggyBac ITR sequence, a second nucleic acid sequence and at least one DNA spacer sequence.

In some aspects, an AAV piggyBac transposon polynucleotide can comprise in the 5′ to 3′ direction a first nucleic acid sequence, a first piggyBac ITR sequence, a first insulator sequence, at least one promoter sequence, at least one transgene sequence, a polyA sequence, a second insulator sequence, a second piggyBac ITR sequence, a second nucleic acid sequence and at least one DNA spacer sequence.

In some aspects, an AAV piggyBac transposon polynucleotide can comprise a first nucleic acid sequence, followed by a first piggyBac ITR sequence, followed by a first insulator sequence, followed by at least one promoter sequence, followed by at least one transgene sequence, followed by a polyA sequence, followed by a second insulator sequence, followed by a second piggyBac ITR sequence, followed by a second nucleic acid sequence and followed by at least one DNA spacer sequence.

An AAV piggyBac transposon polynucleotide can comprise a first nucleic acid sequence, a first piggyBac ITR sequence, a first insulator sequence, at least one promoter sequence, a first transgene sequence, at least one self-cleaving peptide sequence, an at least second transgene sequence, a polyA sequence, a second insulator sequence, a second piggyBac ITR sequence, and a second nucleic acid sequence.

In some aspects, an AAV piggyBac transposon polynucleotide can comprise in the 5′ to 3′ direction a first nucleic acid sequence, a first piggyBac ITR sequence, a first insulator sequence, at least one promoter sequence, a first transgene sequence, at least one self-cleaving peptide sequence, an at least second transgene sequence, a polyA sequence, a second insulator sequence, a second piggyBac ITR sequence, and a second nucleic acid sequence.

In some aspects, an AAV piggyBac transposon polynucleotide can comprise a first nucleic acid sequence, followed by a first piggyBac ITR sequence, followed by a first insulator sequence, followed by at least one promoter sequence, followed by a first transgene sequence, followed by at least one self-cleaving peptide sequence, followed by an at least second transgene sequence, followed by a polyA sequence, followed by a second insulator sequence, followed by a second piggyBac ITR sequence, and followed by a second nucleic acid sequence.

An AAV piggyBac transposon polynucleotide can comprise a first nucleic acid sequence, a first piggyBac ITR sequence, a first insulator sequence, a first promoter sequence, a first transgene sequence, at least a second promoter sequence, an at least second transgene sequence, a polyA sequence, a second insulator sequence, a second piggyBac ITR sequence, and a second nucleic acid sequence.

An AAV piggyBac transposon polynucleotide can comprise in the 5′ to 3′ direction a first nucleic acid sequence, a first piggyBac ITR sequence, a first insulator sequence, a first promoter sequence, a first transgene sequence, an at least a second promoter sequence, an at least second transgene sequence, a polyA sequence, a second insulator sequence, a second piggyBac ITR sequence, and a second nucleic acid sequence.

An AAV piggyBac transposon polynucleotide can comprise a first nucleic acid sequence, followed by a first piggyBac ITR sequence, followed by a first insulator sequence, followed by a first promoter sequence, followed by a first transgene sequence, followed by an at least a second promoter sequence, followed by an at least second transgene sequence, followed by a polyA sequence, followed by a second insulator sequence, followed by a second piggyBac ITR sequence and followed by a second nucleic acid sequence.

In some aspects, an AAV piggyBac transposon polynucleotide can comprise a first nucleic acid sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence and a second nucleic acid sequence.

In some aspects, an AAV piggyBac transposon polynucleotide can comprise in the 5′ to 3′ direction a first nucleic acid sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence and a second nucleic acid sequence.

In some aspects, an AAV piggyBac transposon polynucleotide can comprise a first nucleic acid sequence, followed by a first piggyBac ITR sequence, followed by a first insulator sequence, followed by at least one promoter sequence, followed by a first transgene sequence, followed by a first self-cleaving peptide sequence, followed by a second transgene sequence, followed by an at least second self-cleaving peptide sequence, followed by at least a third transgene sequence, followed by a polyA sequence, followed by a second insulator sequence and followed by a second nucleic acid sequence.

In some aspects, an AAV piggyBac transposon polynucleotide can comprise a first nucleic acid sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence, a second piggyBac ITR sequence and a second nucleic acid sequence.

In some aspects, an AAV piggyBac transposon polynucleotide can comprise in the 5′ to 3′ direction a first nucleic acid sequence, a first piggyBac ITR sequence, a first insulator sequence, at least one promoter sequence, a first transgene sequence, a first self-cleaving peptide sequence, a second transgene sequence, an at least second self-cleaving peptide sequence, at least a third transgene sequence, a polyA sequence, a second insulator sequence, a second piggyBac ITR sequence and a second nucleic acid sequence.

In some aspects, an AAV piggyBac transposon polynucleotide can comprise a first nucleic acid sequence, followed by a first piggyBac ITR sequence, followed by a first insulator sequence, followed by at least one promoter sequence, followed by a first transgene sequence, followed by a first self-cleaving peptide sequence, followed by a second transgene sequence, followed by an at least second self-cleaving peptide sequence, followed by at least a third transgene sequence, followed by a polyA sequence, followed by a second insulator sequence, followed by a second piggyBac ITR sequence and followed by a second nucleic acid sequence.

The present disclosure further provides polynucleotide molecules comprising the same structure as the AAV piggyBac transposon polynucleotides described above except without the first AAV ITR sequence and the second AAV ITR sequence.

### AAV ITR Sequences

In some aspects, an AAV ITR sequence can comprise any AAV ITR sequence known in the art. In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 1, 2, 17 and 18.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 1 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2.

In some aspects, a first AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 17 and a second AAV ITR sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 18.

### First and Second Nucleic Acid Sequences

In some aspects, a first nucleic acid sequence or a second nucleic acid sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 1, 2, 17 and 18.

In some aspects, a first nucleic acid sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 1 and a second nucleic acid sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 2.

In some aspects, a first nucleic acid sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 17 and a second nucleic acid sequence can comprise consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 18.

piggyBac ITR Sequences

In some aspects, a piggyBac ITR sequence can comprise any piggyBac ITR sequence known in the art. In some aspects, a piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 3, 4, 19 and 21.

In some aspects, a piggyBac ITR sequence can comprise any piggyBac ITR sequence known in the art. In some aspects, a piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 1 and 2.

In some aspects, a piggyBac ITR sequence can comprise any piggyBac ITR sequence known in the art. In some aspects, a piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 79 and 80.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 3 and a second piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 4.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 19 and a second piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 21.

In some aspects, a first piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 79 and a second piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 80.

In some aspects of the methods of the present disclosure, a piggyBac ITR sequence, such as a first piggyBac ITR sequence and/or a second piggyBac ITR sequence in an AAV piggyBac transposon can comprise, consist essentially of, or consist of a Sleeping Beauty transposon ITR, a Helraiser transposon ITR, a Tol2 transposon ITR, a TcBuster transposon ITR or any combination thereof.

In some aspects, a piggyBac ITR sequence of the present disclosure can be flanked on either or both ends by at least one of the following sequences: 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. In some aspects, a piggyBac ITR sequence can be flanked by 5′-TTAA-3′. Thus, any AAV transposase polynucleotide, AAV piggyBac transposon polynucleotide and/or any liver nanoplasmid of the present disclosure can further comprise any one of: 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′ flanking a piggyBac ITR sequence.

### Insulator Sequences

In some aspects, an insulator sequence can comprise any insulator sequence known in the art. In some aspects, an insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 5, 6 and 20.

In some aspects, a first insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 5 and a second insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any SEQ ID NO: 6.

In some aspects, a first insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 5 and a second insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any SEQ ID NO: 20.

### Promoter Sequences

In some aspects, a promoter sequence can comprise any promoter sequence known in the art. In some aspects, a promoter sequence can comprise any liver-specific promoter sequence known in the art.

In some aspects, a promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 7-12.

In some aspects, a promoter sequence can comprise an ApoEhAAT promoter sequence. An ApoEhAAT promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 7.

In some aspects, a promoter sequence can comprise a TTR promoter sequence. A TTR promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 8. A TTR promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 9.

In some aspects, a promoter sequence can comprise an HLP promoter sequence. An HLP promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 10.

In some aspects, a promoter sequence can comprise a TTRm promoter sequence. A TTRm promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 11. A TTRm promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 12.

### Transgene Sequences

In some aspects, a transgene sequence can comprise a nucleic acid sequence that encodes for a Factor VIII (FVIII) polypeptide. In some aspects, a transgene sequence can comprise a nucleic acid sequence that encodes for a FVIII polypeptide, wherein the FVIII polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 26. In some aspects, a nucleic acid sequence that encodes for a FVIII polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 13.

In some aspects, a transgene sequence can comprise a nucleic acid sequence that encodes for a Factor IX (FIX) polypeptide. In some aspects, a transgene sequence can comprise a nucleic acid sequence that encodes for a FIX polypeptide, wherein the FIX polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 27. In some aspects, a nucleic acid sequence that encodes for a FIX polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 23.

In some aspects, a transgene sequence can be codon optimized according to methods known in the art.

In some aspects, the nucleic acid sequence encoding a polypeptide (e.g. FVIII, FIX.) can be a codon optimized nucleic acid sequence that encodes for the polypeptide. A codon optimized nucleic acid sequence encoding a polypeptide can comprise, consist essentially of, or consist of a nucleic acid sequence that is no more than 65%, 70%, 75%, 80%, 85%, 90%, 95%, 96%, 97%, 98%, 99% (or any percentage in between) identical to the wildtype human nucleic acid sequence encoding the polypeptide.

SEQ ID NOs: 13 and 23 are unique codon optimized nucleic acid sequences that can be included in the polynucleotides, vectors and compositions of the present disclosure.

In some aspects, a codon optimized nucleic acid sequence encoding a polypeptide, such as those put forth in SEQ ID NOs: 13 and 23, can comprise no donor splice sites. In some aspects, a codon optimized nucleic acid sequence encoding a polypeptide can comprise no more than about one, or about two, or about three, or about four, or about five, or about six, or about seven, or about eight, or about nine, or about ten donor splice sites. In some aspects, a codon optimized nucleic acid sequence encoding a polypeptide comprises at least one, or at least two, or at least three, or at least four, or at least five, or at least six, or at least seven, or at least eight, or at least nine, or at least ten fewer donor splice sites as compared to the wildtype human nucleic acid sequence encoding the polypeptide. Without wishing to be bound by theory, the removal of donor splice sites in the codon optimized nucleic acid sequence can unexpectedly and unpredictably increase expression of the polypeptide in vivo, as cryptic splicing is prevented. Moreover, cryptic splicing may vary between different subjects, meaning that the expression level of the polypeptide comprising donor splice sites may unpredictably vary between different subjects.

In some aspects, a codon optimized nucleic acid sequence encoding a polypeptide, such as those put forth in SEQ ID NOs: 13 and 23, can have a GC content that differs from the GC content of the wildtype human nucleic acid sequence encoding the polypeptide. In some aspects, the GC content of a codon optimized nucleic acid sequence encoding a polypeptide is more evenly distributed across the entire nucleic acid sequence, as compared to the wildtype human nucleic acid sequence encoding the polypeptide. Without wishing to be bound by theory, by more evenly distributing the GC content across the entire nucleic acid sequence, the codon optimized nucleic acid sequence exhibits a more uniform melting temperature (“Tm”) across the length of the transcript. The uniformity of melting temperature results unexpectedly in increased expression of the codon optimized nucleic acid in a human subject, as transcription and/or translation of the nucleic acid sequence occurs with less stalling of the polymerase and/or ribosome.

In some aspects, the codon optimized nucleic acid sequence encoding a polypeptide, such as those put forth in SEQ ID NOs: 13 and 23, exhibits at least 5%, at least 10%, at least 20%, at least 30%, at least 50%, at least 75%, at least 100%, at least 200%, at least 300%, at least 500%, or at least 1000% increased expression in a human subject relative to a wild-type or non-codon optimized nucleic acid sequence encoding the polypeptide.

In some aspects, an at least one transgene sequence can be operatively linked to at least one promoter sequence present in the same polynucleotide.

polyA Sequences

In some aspects, a polyA sequence can comprise any polyA sequence known in the art. Non-limiting examples of polyA sequences include, but are not limited to, SV40 polyA sequences In some aspects, an insulator sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 14 and 22.

### Self-Cleaving Peptide Sequence

In some aspects, a self-cleaving peptide sequence can comprise any self-cleaving peptide sequence known in the art. In some aspects, a self-cleaving peptide sequence can comprise an 2A self-cleaving peptide sequence known in the art. Non-limiting examples of self-cleaving peptides include a T2A peptide, GSG-T2A peptide, an E2A peptide, a GSG-E2A peptide, an F2A peptide, a GSG-F2A peptide, a P2A peptide, or a GSG-P2A peptide.

In some aspects, a self-cleaving peptide sequence can comprise a nucleic acid sequence that encodes for a T2A peptide. In some aspects, a nucleic acid sequence that encodes for a T2A peptide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 15.

In some aspects, a self-cleaving peptide sequence can comprise a nucleic acid sequence that encodes for a GSG-T2A peptide. In some aspects, a nucleic acid sequence that encodes for a GSG-T2A peptide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 24.

### DNA Spacer Sequences

In some aspects, a DNA spacer sequence can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 28.

DNA spacer sequences can be located at any position within an AAV piggyBac transposon polynucleotide or an AAV piggyBac transposase polynucleotide.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 29.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 30.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 31.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 32.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 33.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 34.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 35.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 36.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 37.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 38.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 39.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 40.

In some aspects, a AAV piggyBac transposon polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to SEQ ID NO: 41.

The present disclosure also provides a polynucleotide can comprising, consisting essentially of or consisting of a nucleic acid sequence at least 65%, 70%, 75%, 80%, 85%, 90%, 95%, 96%, 97%, 98%, 99% or 100% (or any percentage in between) identical to any one of SEQ ID NOs: 29-41.

### AAV Transposase Polynucleotides

The present disclosure provides compositions comprising AAV transposase polynucleotides.

In some aspects an AAV transposase polynucleotide can comprise at least one AAV inverted terminal repeat (ITR) sequence. In some aspects an AAV transposase polynucleotide can comprise at least one promoter sequence. In some aspects, an AAV transposase polynucleotide can comprise at least one transposase sequence. In some aspects, an AAV transposon polynucleotide can comprise at least one polyA sequence. In some aspects, an AAV transposon polynucleotide can comprise at least one DNA spacer sequence.

In some aspects, an AAV transposase polynucleotide can comprise a first AAV ITR sequence, at least one promoter sequence, at least one transposase sequence, a polyA sequence and a second AAV ITR sequence.

In some aspects, an AAV transposase polynucleotide can comprise in the 5′ to 3′ direction a first AAV ITR sequence, at least one promoter sequence, at least one transposase sequence, a polyA sequence and a second AAV ITR sequence.

In some aspects, an AAV transposase polynucleotide can comprise a first AAV ITR sequence, followed by at least one promoter sequence, followed by at least one transposase sequence, followed by a polyA sequence and followed by a second AAV ITR sequence.

In some aspects, an AAV transposase polynucleotide can comprise a first AAV ITR sequence, at least one promoter sequence, at least one transposase sequence, a polyA sequence, at least one DNA spacer sequence and a second AAV ITR sequence.

In some aspects, an AAV transposase polynucleotide can comprise in the 5′ to 3′ direction a first AAV ITR sequence, at least one promoter sequence, at least one transposase sequence, a polyA sequence, at least one DNA spacer sequence and a second AAV ITR sequence.

In some aspects, an AAV transposase polynucleotide can comprise a first AAV ITR sequence, followed by at least one promoter sequence, followed by at least one transposase sequence, followed by a polyA sequence, followed by at least one DNA spacer sequence and followed by a second AAV ITR sequence.

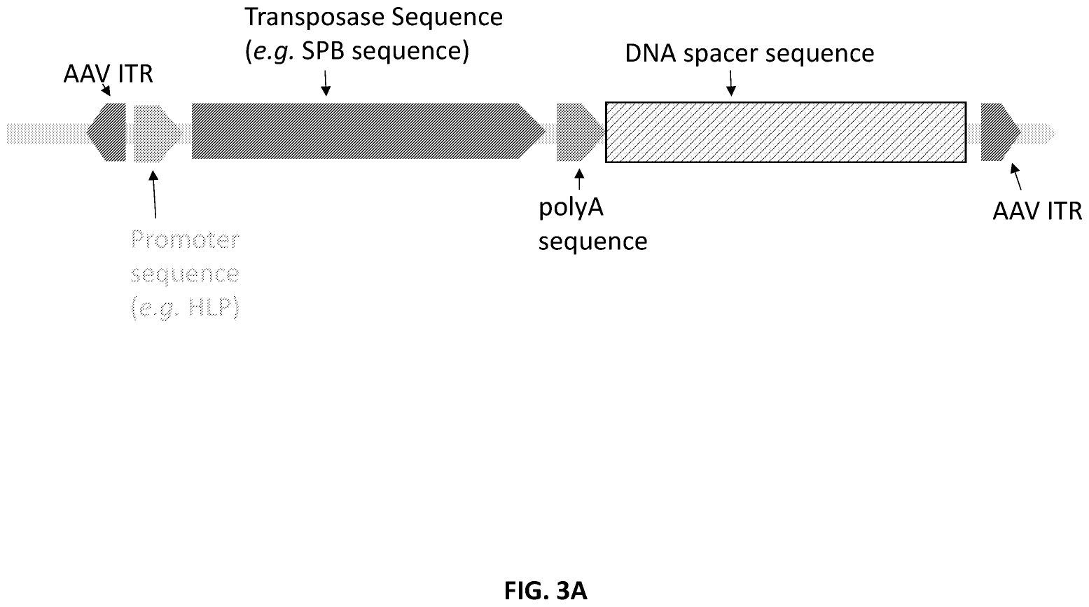

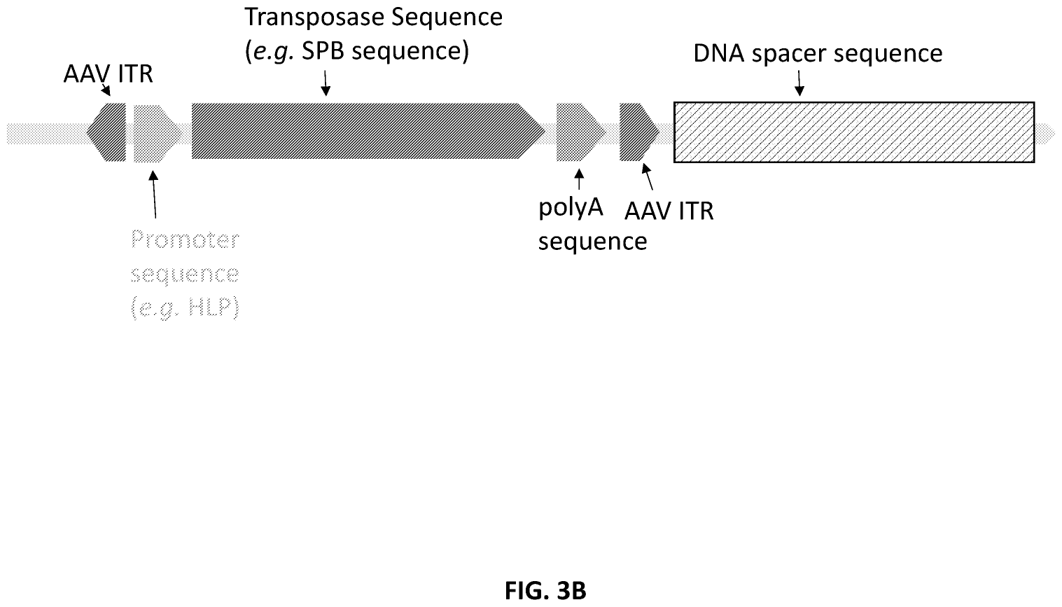

In a non-limiting example of the preceding AAV transposase polynucleotides, the at least one promoter sequence can comprise a hybrid liver promoter (HLP) and the at least one transposase sequence can comprise a nucleic acid sequence encoding a Super piggyBac™ (SPB) transposase polypeptide. This non-limiting example of an AAV piggyBac transposon polynucleotide is shown in FIG. 3A.

In some aspects, an AAV transposase polynucleotide can comprise, in between a polyA sequence and a second AAV ITR sequence, at least one DNA spacer sequence, as is shown in the non-limiting example presented in FIG. 3A.

In some aspects, an AAV transposase polynucleotide can comprise a first AAV ITR sequence, at least one promoter sequence, at least one transposase sequence, a polyA sequence, a second AAV ITR sequence and at least one DNA spacer sequence.

In some aspects, an AAV transposase polynucleotide can comprise in the 5′ to 3′ direction a first AAV ITR sequence, at least one promoter sequence, at least one transposase sequence, a polyA sequence, a second AAV ITR sequence and at least one DNA spacer sequence.

In some aspects, an AAV transposase polynucleotide can comprise a first AAV ITR sequence, followed by at least one promoter sequence, followed by at least one transposase sequence, followed by a polyA sequence, followed by a second AAV ITR sequence and followed by at least one DNA spacer sequence.

In a non-limiting example of the preceding AAV transposase polynucleotides, the at least one promoter sequence can comprise a hybrid liver promoter (HLP) and the at least one transposase sequence can comprise a nucleic acid sequence encoding a Super piggyBac™ (SPB) transposase polypeptide. This non-limiting example of an AAV piggyBac transposon polynucleotide is shown in FIG. 3B.

In some aspects, an AAV transposase polynucleotide can comprise, after a second AAV ITR sequence, at least one DNA spacer sequence, as is shown in the non-limiting example presented in FIG. 3B.

In some aspects, an AAV transposase polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 42.

In some aspects, an AAV transposase polynucleotide can comprise, consist essentially of or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 43.

### Transposase Sequences

In some aspects, a transposase sequence can comprise a nucleic acid sequence that encodes for any transposase polypeptide known in the art. In some aspects, a transposase sequence can comprise a nucleic acid sequence that encodes for a piggyBac™ (PB) transposase polypeptide. In some aspects, a transposase sequence can comprise a nucleic acid sequence that encodes for a piggyBac-like (PBL) transposase polypeptide. In some aspects, a transposase sequence can comprise a nucleic acid sequence that encodes for a Super piggyBac™ (SPB) transposase polypeptide.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3′, 5′-CCCA-3′, 5′-CGTA-3′, 5′-GTCC-3′, 5′-TAAG-3′, 5′-TCTA-3′, 5′-TGAG-3′, 5′-TGTT-3′, 5′-TTCA-3′5′-TTCT-3′ and 5′-TTTT-3′. The PB or PBL transposon system has no payload limit for the genes of interest that can be included between the ITRs.

Exemplary amino acid sequences for one or more PB, PBL and SPB transposases are disclosed in U.S. Pat. Nos. 6,218,185; 6,962,810 and 8,399,643. In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 44.

The PB or PBL transposase can comprise or consist of an amino acid sequence having an amino acid substitution at two or more, at three or more or at each of positions 30, 165, 282, and/or 538 of the sequence of SEQ ID NO: 44. The transposase can be a SPB transposase that comprises or consists of the amino acid sequence of the sequence of SEQ ID NO: 44 wherein the amino acid substitution at position 30 can be a substitution of a valine (V) for an isoleucine (I), the amino acid substitution at position 165 can be a substitution of a serine (S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 45.

In certain aspects wherein the transposase comprises the above-described mutations at positions 30, 165, 282 and/or 538, the PB, PBL and SPB transposases can further comprise an amino acid substitution at one or more of positions 3, 46, 82, 103, 119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 44 or SEQ ID NO: 45 are described in more detail in PCT Publication No. WO 2019/173636 and PCT/US2019/049816.

In a preferred aspect, the PB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 46.

The PB or PBL transposase can comprise or consist of an amino acid sequence having an amino acid substitution at two or more, at three or more or at each of positions 29, 164, 281, and/or 537 of the sequence of SEQ ID NO: 46. The transposase can be a SPB transposase that comprises or consists of the amino acid sequence of the sequence of SEQ ID NO: 46 wherein the amino acid substitution at position 29 can be a substitution of a valine (V) for an isoleucine (I), the amino acid substitution at position 164 can be a substitution of a serine (S) for a glycine (G), the amino acid substitution at position 281 can be a substitution of a valine (V) for a methionine (M), and the amino acid substitution at position 537 can be a substitution of a lysine (K) for an asparagine (N). In a preferred aspect, the SPB transposase comprises or consists of an amino acid sequence at least 65%, 70%, 75%, 80%, 85%, 90%, 95%, 96%, 97%, 98%, 99% or 100% (or any percentage in between) identical to SEQ ID NO: 47.

In certain aspects wherein the transposase comprises the above-described mutations at positions 29, 164, 281, and/or 537, the PB, PBL and SPB transposases can further comprise an amino acid substitution at one or more of positions 2, 45, 81, 102, 118, 124, 176, 179, 184, 186, 199, 206, 208, 225, 234, 239, 240, 242, 257, 295, 297, 310, 314, 318, 326, 327, 339, 420, 435, 455, 469, 485, 502, 551, 569 and 590 of the sequence of SEQ ID NO: 46 or SEQ ID NO: 47 are described in more detail in PCT Publication No. WO 2019/173636 and PCT/US2019/049816.

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, a PB, PBL or SPB transposase can fused to a nuclear localization signal. Examples of PB, PBL or SPB transposases fused to a nuclear localization signal are disclosed in U.S. Pat. Nos. 6,218,185; 6,962,810, 8,399,643 and WO 2019/173636. A nuclear localization signal can comprise, consist essentially of or consist of a of the amino acid sequence at least 65%, 70%, 75%, 80%, 85%, 90%, 95%, 96%, 97%, 98%, 99% or 100% (or any percentage in between) identical to SEQ ID NO: 48. A nuclear localization signal can be encoded by a nucleic acid sequence that comprises, consists essentially of or consists of the nucleic acid sequence at least 65%, 70%, 75%, 80%, 85%, 90%, 95%, 96%, 97%, 98%, 99% or 100% (or any percentage in between) identical to SEQ ID NO: 49.

In some aspects, a nuclear localization signal can be fused to a PB, PBL or SPB transposase using a G4S linker located between the NLS and the PB, PBL or SPB. A G4S linker can comprise, consist essentially of or consist of an amino acid sequence at least 65%, 70%, 75%, 80%, 85%, 90%, 95%, 96%, 97%, 98%, 99% or 100% (or any percentage in between) identical to SEQ ID NO: 50. A G4S linker can be encoded by a nucleic acid sequence that comprises, consists essentially of or consists of the nucleic acid sequence at least 65%, 70%, 75%, 80%, 85%, 90%, 95%, 96%, 97%, 98%, 99% or 100% (or any percentage in between) identical to SEQ ID NO: 51.

In some aspects, a transposase sequence can comprise a nucleic acid sequence that encodes for a SBP transposase polypeptide fused to an NLS, wherein the SBP transposase polypeptide fused to an NLS comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 52. In some aspects, a nucleic acid sequence that encodes for a SBP transposase polypeptide fused to an NLS can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 53.

In some aspects, a transposase sequence can comprise a nucleic acid sequence that encodes for a SBP transposase polypeptide fused to an NLS, wherein the SBP transposase polypeptide fused to an NLS comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 54. In some aspects, a nucleic acid sequence that encodes for a SBP transposase polypeptide fused to an NLS can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 55.

In some aspects, a transposase sequence can comprise a nucleic acid sequence that encodes for a Sleeping Beauty transposase polypeptide (for example as disclosed in U.S. Pat. No. 9,228,180). In some aspects, a transposase sequence can comprise a nucleic acid sequence that encodes for a Hyperactive Sleeping Beauty (SB100X) transposase polypeptide. In some aspects, a Sleeping Beauty transposase comprises or consists of an amino acid sequence at least 65%, 70%, 75%, 80%, 85%, 90%, 95%, 96%, 97%, 98%, 99% or 100% (or any percentage in between) identical to SEQ ID NO: 56 or 57. In a preferred aspect, hyperactive Sleeping Beauty (SB100X) transposase comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NO: 58 or 59.

In some aspects, a transposase sequence can comprise a nucleic acid sequence that encodes for a helitron transposase polypeptide (for example, as disclosed in WO 2019/173636). In some aspects, a Helitron transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NO: 60 or 61.

In some aspects, a transposase sequence can comprise a nucleic acid sequence that encodes for a Tol2 transposase polypeptide (for example, as disclosed in WO 2019/173636). In some aspects, a Tol2 transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NO: 62 or 63.

In some aspects, a transposase sequence can comprise a nucleic acid sequence that encodes for a TcBuster transposase polypeptide (for example, as disclosed in WO 2019/173636) or a mutant TcBuster transposase polypeptide (as described in more detail in PCT Publication No. WO 2019/173636 and PCT/US2019/049816). In some aspects, a TcBuster transposase polypeptide comprises, consists essentially of or consists of an amino acid sequence at least 65%, 70%, 75%, 80%, 85%, 90%, 95%, 96%, 97%, 98%, 99% or 100% (or any percentage in between) identical to SEQ ID NO: 64 or 65. The polynucleotide encoding a TcBuster transposase can comprise or consist of a naturally occurring nucleic acid sequence or a non-naturally occurring nucleic acid sequence.

In some aspects of the present disclosure, a transgene sequence can comprise a luciferase sequence.

In some aspects, a luciferase sequence can comprise a nucleic acid sequence that encodes for a nanoluciferase (nLuc) polypeptide. In some aspects, a nucleic acid sequence that encodes for an nLuc polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the sequences put forth in SEQ ID NOs: 16 and 25.

### Vectors of the Present Disclosure

The present disclosure provides compositions comprising a vector, wherein the vector comprises at least one adeno-associated virus (AAV) piggyBac transposon polynucleotide. A vector comprising at least one adeno-associated virus (AAV) piggyBac transposon polynucleotide is herein referred to as an “AAV piggyBac transposon vector”.

The present disclosure provides compositions comprising a vector, wherein the vector comprises at least one AAV transposase polynucleotide. A vector comprising at least one AAV transposase polynucleotide is herein referred to as an “AAV transposase vector”.

A vector of the present disclose can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV).

Exemplary adeno-associated viruses and recombinant adeno-associated viruses include, but are not limited to all serotypes (e.g., AAV1, AAV2, AAV3, AAV4, AAV5, AAV6, AAV7, AAV8, AAV9, AAV10 and AAV 11). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (also referred to as AAV-KP1; described in detail in Kerun et al. JCI Insight, 2019; 4(22):e131610) and AAV-NP59 (described in detail in Paulk et al. Molecular Therapy, 2018; 26(1): 289-303).

The present disclosure provides a composition comprising a plurality of AAV-KP-1 particles comprising at least one adeno-associated virus (AAV) piggyBac transposon polynucleotide. The present disclosure provides a composition comprising a plurality of AAV-KP-1 particles comprising at least one AAV transposase polynucleotide. The present disclosure provides a composition comprising a plurality of AAV-KP-1 particles comprising at least one adeno-associated virus (AAV) piggyBac transposon polynucleotide and a plurality of AAV-KP-1 particles comprising at least one AAV transposase polynucleotide.

The present disclosure provides a composition comprising a plurality of AAV-NP59 particles comprising at least one adeno-associated virus (AAV) piggyBac transposon polynucleotide. The present disclosure provides a composition comprising a plurality of AAV-NP59 particles comprising at least one AAV transposase polynucleotide. The present disclosure provides a composition comprising a plurality of AAV-NP59 particles comprising at least one adeno-associated virus (AAV) piggyBac transposon polynucleotide and a plurality of AAV-NP59 particles comprising at least one AAV transposase polynucleotide.

The viral vectors and viral particles of the present disclosure can be produced using standard methods known in the art.

In some aspects, AAV-KP-1 particles of the present disclosure can be produced using a KP-1 capsid vector, wherein the KP-1 capsid vector comprises at least one of the nucleic acid sequences of SEQ ID NO: 66 and SEQ ID NO: 67. In some aspects, AAV-KP-1 particles of the present disclosure can be produced using an AAV vector packaging plasmid, wherein the AAV vector packaging plasmid comprises at least of the nucleic acid sequences of SEQ ID NO: 68 and SEQ ID NO: 69

In some aspects, AAV-NP59 particles of the present disclosure can be produced using a NP-59 capsid vector, wherein the NP-59 capsid vector comprises at least one of the nucleic acid sequences of SEQ ID NO: 70, SEQ ID NO: 71 and SEQ ID NO: 72. In some aspects, AAV-NP59 particles of the present disclosure can be produced using an AAV vector packaging plasmid, wherein the AAV vector packaging plasmid comprises at least of the nucleic acid sequences of SEQ ID NO: 68 and SEQ ID NO: 69.

The cell delivery compositions (e.g., polynucleotides, vectors) disclosed herein can comprise a nucleic acid encoding a therapeutic protein or therapeutic agent. Examples of therapeutic proteins include those disclosed in PCT Publication No. WO 2019/173636 and PCT/US2019/049816. Therapeutic proteins can also include, but are not limited to, any one of polypeptides described herein as part of transgene sequences (e.g. FVIII, FIX, etc.)

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994,), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods.

Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are described in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 μm. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

An effective amount can comprise an amount of about 0.001 to about 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

In aspects where the compositions to be administered to a subject in need thereof are modified cells as disclosed herein, the cells can be administered between about 1×10³ and 1×10¹⁵ cells; about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In one aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/049816.

### Methods of Using the Compositions of the Disclosure

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method for treating hemophilia in a subject in need thereof comprising administering to the subject at least one therapeutically effective amount of at least one composition of the present disclosure.

The present disclosure provides at least one composition of the present disclosure for the use in the treatment of hemophilia in a subject, wherein the at least one composition is for administration to the subject in at least one therapeutically effective amount.

The present disclosure provides the use of at least one composition of the present disclosure for the manufacture of a medicament for the treatment of hemophilia in a subject, wherein the at least one composition is for administration to the subject in at least one therapeutically effective amount.

In some aspects of the preceding methods and uses, the at least one composition of the present disclosure can comprise at least one AAV piggyBac transposon vector of the present disclosure.

Accordingly, the disclosure provides a method for treating hemophilia in a subject in need thereof comprising administering to the subject at least one therapeutically effective amount of at least one AAV piggyBac transposon vector of the present disclosure.

The present disclosure provides at least one AAV piggyBac transposon vector of the present disclosure for the use in the treatment of hemophilia in a subject, wherein the at least one AAV piggyBac transposon vector is for administration to the subject in at least one therapeutically effective amount.

The present disclosure provides the use of at least one AAV piggyBac transposon vector of the present disclosure for the manufacture of a medicament for the treatment of hemophilia in a subject, wherein the at least one AAV piggyBac transposon vector is for administration to the subject in at least one therapeutically effective amount.

In some aspects of the preceding methods and uses, the at least one composition of the present disclosure can comprise at least one AAV transposase vector of the present disclosure.

Accordingly, the disclosure provides a method for treating hemophilia in a subject in need thereof comprising administering to the subject at least one therapeutically effective amount of at least one AAV transposase vector of the present disclosure.

The present disclosure provides at least one AAV transposase vector of the present disclosure for the use in the treatment of hemophilia in a subject, wherein the at least one AAV transposase vector is for administration to the subject in at least one therapeutically effective amount.

The present disclosure provides the use of at least one AAV transposase vector of the present disclosure for the manufacture of a medicament for the treatment of hemophilia in a subject, wherein the at least one AAV transposase vector is for administration to the subject in at least one therapeutically effective amount.

The present disclosure provides methods of treating hemophilia in a subject, the methods comprising administering to the subject: a) at least one therapeutically effective amount of a composition comprising a nucleic acid molecule comprising a transposon, wherein the transposon comprises a nucleotide sequence encoding at least one therapeutic protein; and b) at least one therapeutically effective amount of a composition comprising a nucleic acid molecule comprising a nucleotide sequence encoding at least one transposase.

In some aspects of the preceding method, a composition comprising a nucleic acid molecule comprising a transposon can be any AAV piggyBac transposon vector described herein.

In some aspects of the preceding method, a composition comprising a nucleic acid molecule comprising a nucleotide sequence encoding at least one transposase can be any AAV transposase vector of the present disclosure.

Accordingly, the present disclosure provides methods of treating hemophilia in a subject, the methods comprising administering to the subject: a) at least one therapeutically effective amount of at least one AAV piggyBac transposon vector of the present disclosure; and b) at least one therapeutically effective amount of at least one AAV transposase vector of the present disclosure.

Accordingly the present disclosure provides a combination of at least one AAV piggyBac transposon vector of the present disclosure and at least one AAV transposase vector of the present disclosure for use in in the treatment of hemophilia in a subject, wherein the at least one AAV piggyBac transposon vector is for administration to the subject in at least one therapeutically effective amount, and wherein the at least one AAV transposase vector is for administration to the subject in at least one therapeutically effective amount.

Accordingly the present disclosure provides the use of a combination of at least one AAV piggyBac transposon vector of the present disclosure and at least one AAV transposase vector of the present disclosure in the manufacture of a medicament for the treatment of hemophilia in a subject, wherein the at least one AAV piggyBac transposon vector is for administration to the subject in at least one therapeutically effective amount, and wherein the at least one AAV transposase vector is for administration to the subject in at least one therapeutically effective amount.

In some aspects, the hemophilia can be hemophilia A. In some aspects, the hemophilia can be hemophilia B. In some aspects, the hemophilia can be hemophilia C. In some aspects, the hemophilia can be Parahemophilia.

In some aspects of the preceding methods, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered concurrently. In some aspects, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered sequentially. In some aspects, a composition comprising a nucleic acid molecule comprising a transposon, wherein the transposon comprises a nucleotide sequence encoding at least one therapeutic protein and a composition comprising a nucleic acid molecule comprising a nucleotide sequence encoding at least one transposase can be administered in temporal proximity.

As used herein, the term “temporal proximity” refers to that administration of one therapeutic composition (e.g., a composition comprising a transposon) occurs within a time period before or after the administration of another therapeutic composition (e.g., a composition comprising a transposase), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In some aspects of the treatment methods of the present disclosure, the administration of the at least one composition and/or vector of the present disclosure to a subject can result in the expression of an exogenous protein (e.g. a therapeutic protein, a transposase, etc.) in at least one organ and/or tissue and/or bodily fluid in the subject.

In some aspects, the administration of the at least one composition and/or vector of the present disclosure results in the expression of the exogenous protein in at least about 10%, or at least about 15%, or at least bout 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of the cells in the tissue and/or organ and/or bodily fluid.

In some aspects, the administration of the at least one composition and/or vector of the present disclosure results in the expression of the exogenous protein in at least about 10%, or at least about 15%, or at least bout 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99% of a specific subset or subsets of cells in the tissue and/or organ and/or bodily fluid.

In some aspects, the administration of the at least one composition and/or vector of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in the tissue and/or organ and/or bodily fluid.

In some aspects, the administration of the at least one composition and/or vector of the present disclosure results in the expression of the exogenous protein for at least about 1 day, or at least about 2 days, or at least about 3 days, or at least about 4 days, or at least about 5 days, or at least about 6 days, or at least about 7 days, or at least about 8 days, or at least about 9 days, or at least about 10 days in a specific subset or subsets of cells in the tissue and/or organ and/or bodily fluid.

In some aspects, the administration of the at least one composition and/or vector of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in the tissue and/or organ and/or bodily fluid.

In some aspects, the administration of the at least one composition and/or vector of the present disclosure results in the expression of the exogenous protein for no more than about 1 day, or no more than about 2 days, or no more than about 3 days, or no more than about 4 days, or no more than about 5 days, or no more than about 6 days, or no more than about 7 days, or no more than about 8 days, or no more than about 9 days, or no more than about 10 days in a specific subset or subsets of cells in the tissue and/or organ and/or bodily fluid.

In some aspects, the tissue and/or organ and/or bodily fluid can be the liver. In some aspects, the specific subset or subsets of cells can include, but are not limited to, hepatocytes, a hepatic stellate cells, Kupffer cells, liver sinusoidal endothelial cells or any combination thereof.

In some aspects, the tissue and/or organ and/or bodily fluid can be blood, plasma, serum or any combination thereof.

Any method of the present disclosure can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one additional treatment for urea cycle disorders.

### Exemplary Embodiments of the Present Disclosure

Embodiment 1a. An adeno-associated virus (AAV) piggyBac transposon polynucleotide comprising in the 5′ to 3′ direction:
a) a first AAV inverted terminal repeat (ITR) sequence;
b) a first piggyBac ITR sequence;
c) a first insulator sequence;
d) at least one promoter sequence;
e) at least one transgene sequence;
f) a polyA sequence;
g) a second insulator sequence;
h) a second piggyBac ITR sequence; and
i) a second AAV ITR sequence.

Embodiment 1b. An adeno-associated virus (AAV) piggyBac transposon polynucleotide comprising in the 5′ to 3′ direction:
a) a first nucleic acid sequence;
b) a first piggyBac ITR sequence;
c) a first insulator sequence;
d) at least one promoter sequence;
e) at least one transgene sequence;
f) a polyA sequence;
g) a second insulator sequence;
h) a second piggyBac ITR sequence; and
i) a second nucleic acid sequence.

Embodiment 2. The AAV piggyBac transposon polynucleotide of embodiment 1a or 1b, wherein the AAV piggyBac transposon polynucleotide comprises DNA, cDNA, gDNA, RNA, mRNA or any combination thereof.

Embodiment 3a. The AAV piggyBac transposon polynucleotide of any one of the preceding embodiments, wherein the first and/or the second AAV ITR sequence comprises the nucleic acid sequence of any one of SEQ ID NOs: 1, 2, 17 and 18.

Embodiment 3b. The AAV piggyBac transposon polynucleotide of any one of the preceding embodiments, wherein the first and/or the second nucleic acid sequence comprises the nucleic acid sequence of any one of SEQ ID NOs: 1, 2, 17 and 18.

Embodiment 4a. The AAV piggyBac transposon polynucleotide of any one of the preceding embodiments, wherein the first AAV ITR sequence comprises the nucleic acid sequence of SEQ ID NO: 1 and the second AAV ITR sequence comprises the nucleic acid sequence of SEQ ID NO: 2.

Embodiment 4b. The AAV piggyBac transposon polynucleotide of any one of the preceding embodiments, wherein the first nucleic acid sequence comprises the nucleic acid sequence of SEQ ID NO: 1 and the second nucleic acid sequence comprises the nucleic acid sequence of SEQ ID NO: 2.

Embodiment 5a. The AAV piggyBac transposon polynucleotide of any one of the preceding embodiments, wherein the first AAV ITR sequence comprises the nucleic acid sequence of SEQ ID NO: 17 and the second AAV ITR sequence comprises the nucleic acid sequence of SEQ ID NO: 18.

Embodiment 5b. The AAV piggyBac transposon polynucleotide of any one of the preceding embodiments, wherein the first nucleic acid sequence comprises the nucleic acid sequence of SEQ ID NO: 17 and the second nucleic acid sequence comprises the nucleic acid sequence of SEQ ID NO: 18.

Embodiment 6a. The AAV piggyBac transposon polynucleotide of any one of the preceding embodiments, wherein the first piggyBac ITR sequence and/or the second piggyBac ITR sequence comprises the nucleic acid sequence of any one of SEQ ID NOs: 3, 4, 19 and 21.

Embodiment 6b. The AAV piggyBac transposon polynucleotide of any one of the preceding embodiments, wherein the first piggyBac ITR sequence and/or the second piggyBac ITR sequence comprises the nucleic acid sequence of any one of SEQ ID NOs: 1 and 2.

Embodiment 6c. The AAV piggyBac transposon polynucleotide of any one of the preceding embodiments, wherein the first piggyBac ITR sequence and/or the second piggyBac ITR sequence comprises the nucleic acid sequence of any one of SEQ ID NOs: 79 and 80.

Embodiment 7. The AAV piggyBac transposon polynucleotide of any one of the preceding embodiments, wherein the first piggyBac ITR sequence comprises the nucleic acid sequence of SEQ ID NO: 3 and the second piggyBac ITR comprises the nucleic acid sequence of SEQ ID NO: 4.

Embodiment 8a. The AAV piggyBac transposon polynucleotide of any one of the preceding embodiments, wherein the first piggyBac ITR sequence comprises the nucleic acid sequence of SEQ ID NO: 19 and the second piggyBac ITR comprises the nucleic acid sequence of SEQ ID NO: 21.

Embodiment 8b. The AAV piggyBac transposon polynucleotide of any one of the preceding embodiments, wherein the first piggyBac ITR sequence comprises the nucleic acid sequence of SEQ ID NO: 79 and the second piggyBac ITR comprises the nucleic acid sequence of SEQ ID NO: 80.

Embodiment 9. The AAV piggyBac transposon polynucleotide of any one of the preceding embodiments, wherein the first insulator sequence and/or the second insulator sequence comprises the nucleic acid sequence of any one of SEQ ID NOs: 5, 6 and 20.

Embodiment 10. The AAV piggyBac transposon polynucleotide of any one of the preceding embodiments, wherein the first insulator sequence comprises the nucleic acid sequence of SEQ ID NO: 5 and the second insulator sequence comprises the nucleic acid sequence of SEQ ID NO: 6.

Embodiment 11. The AAV piggyBac transposon polynucleotide of any one of the preceding embodiments, wherein the first insulator sequence comprises the nucleic acid sequence of SEQ ID NO: 5 and the second insulator sequence comprises the nucleic acid sequence of SEQ ID NO: 20.

Embodiment 12. The AAV piggyBac transposon polynucleotide of any one of the preceding embodiments, wherein the at least one promoter sequence comprises the nucleic acid sequence of any one of SEQ IDS NOs: 7-12.

Embodiment 13. The AAV piggyBac transposon polynucleotide of any one of the preceding embodiments, wherein the at least one promoter sequence comprises the nucleic acid sequence of SEQ IDS NO: 7.

Embodiment 14. The AAV piggyBac transposon polynucleotide of any one of the preceding embodiments, wherein the at least one promoter sequence comprises the nucleic acid sequence of SEQ IDS NO: 8.

Embodiment 15. The AAV piggyBac transposon polynucleotide of any one of the preceding embodiments, wherein the at least one promoter sequence comprises the nucleic acid sequence of SEQ IDS NO: 9.

Embodiment 16. The AAV piggyBac transposon polynucleotide of any one of the preceding embodiments, wherein the at least one promoter sequence comprises the nucleic acid sequence of SEQ IDS NO: 10.

Embodiment 17. The AAV piggyBac transposon polynucleotide of any one of the preceding embodiments, wherein the at least one promoter sequence comprises the nucleic acid sequence of SEQ IDS NO: 11.

Embodiment 18. The AAV piggyBac transposon polynucleotide of any one of the preceding embodiments, wherein the at least one promoter sequence comprises the nucleic acid sequence of SEQ IDS NO: 12.

Embodiment 19. The AAV piggyBac transposon polynucleotide of any one of the preceding embodiments, wherein the at least one transgene sequence comprises a nucleic acid sequence encoding a Factor VIII (FVIII) polypeptide.

Embodiment 20. The AAV piggyBac transposon polynucleotide of any one of the preceding embodiments, wherein the FVIII polypeptide comprises the amino acid sequence of SEQ ID NO: 26.

Embodiment 21. The AAV piggyBac transposon polynucleotide of any one of the preceding embodiments, wherein the nucleic acid sequence encoding a FVIII polypeptide comprises the nucleic acid sequence of SEQ ID NO: 13.

Embodiment 22. The AAV piggyBac transposon polynucleotide of any one of the preceding embodiments, wherein the at least one transgene sequence comprises a nucleic acid sequence encoding a Factor IX (FIX) polypeptide.

Embodiment 23. The AAV piggyBac transposon polynucleotide of any one of the preceding embodiments, wherein the FIX polypeptide comprises the amino acid sequence of SEQ ID NO: 27.

Embodiment 24. The AAV piggyBac transposon polynucleotide of any one of the preceding embodiments, wherein the nucleic acid sequence encoding a FIX polypeptide comprises the nucleic acid sequence of SEQ ID NO: 23.

Embodiment 25. The AAV piggyBac transposon polynucleotide of any one of the preceding embodiments, wherein the at least one transgene sequence is operatively linked to the at least one promoter sequence.

Embodiment 26. The AAV piggyBac transposon polynucleotide of any one of the preceding embodiments, wherein the expression of the at least one transgene sequence is controlled by the at least one promoter sequence.

Embodiment 27. The AAV piggyBac transposon polynucleotide of any one of the preceding embodiments, wherein the polyA sequence comprises the nucleic acid sequence of SEQ ID NO: 14.

Embodiment 28. The AAV piggyBac transposon polynucleotide of any one of the preceding embodiments, wherein the polyA sequence comprises the nucleic acid sequence of SEQ ID NO: 22.

Embodiment 29. The AAV piggyBac transposon polynucleotide of any one of the preceding embodiments, wherein the AAV piggyBac transposon polynucleotide further comprises at least a second transgene sequence.

Embodiment 30. The AAV piggyBac transposon polynucleotide of any one of the preceding embodiments, wherein the at least second transgene sequence comprises a luciferase sequence.

Embodiment 31. The AAV piggyBac transposon polynucleotide of any one of the preceding embodiments, wherein the luciferase sequence comprises SEQ ID NO: 16 or 25.

Embodiment 32. The AAV piggyBac transposon polynucleotide of any one of the preceding embodiments, wherein the AAV piggyBac transposon polynucleotide further comprises at least a second promoter sequence.

Embodiment 33. The AAV piggyBac transposon polynucleotide of any one of the preceding embodiments, wherein the at least second promoter sequence is located between the at least one transgene sequence and the at least second transgene sequence.

Embodiment 34. The AAV piggyBac transposon polynucleotide of any one of the preceding embodiments, wherein the AAV piggyBac transposon polynucleotide further comprises at least one self-cleaving peptide sequence, wherein the at least one self-cleaving peptide sequence is a nucleic acid sequence encoding for a T2A peptide, GSG-T2A peptide, an E2A peptide, a GSG-E2A peptide, an F2A peptide, a GSG-F2A peptide, a P2A peptide, or a GSG-P2A peptide.

Embodiment 35. The AAV piggyBac transposon polynucleotide of any one of the preceding embodiments, wherein the at least one self-cleaving peptide sequence is located between the at least one transgene sequence and the at least second transgene sequence.

Embodiment 36. The AAV piggyBac transposon polynucleotide of any one of the preceding embodiments, wherein the AAV piggyBac transposon polynucleotide comprises at least two transgene sequences.

Embodiment 37. The AAV piggyBac transposon polynucleotide of any one of the preceding embodiments, wherein the at least two transgene sequences are the same sequence.

Embodiment 38. The AAV piggyBac transposon polynucleotide of any one of the preceding embodiments, wherein the at least two transgene sequences are different sequences.

Embodiment 39. The AAV piggyBac transposon polynucleotide of any one of the preceding embodiments, further comprising at least one DNA spacer sequence.

Embodiment 40. The AAV piggyBac transposon polynucleotide of any one of the preceding embodiments, wherein the at least one DNA spacer sequence comprises the nucleic acid sequence of SEQ ID NO: 28.

Embodiment 41. The AAV piggyBac transposon polynucleotide of any one of the preceding embodiments, wherein the AAV piggyBac transposon polynucleotide comprises at least two promoter sequences.

Embodiment 42. The AAV piggyBac transposon polynucleotide of any one of the preceding embodiments, wherein the at least two promoter sequences are the same sequence.

Embodiment 43. The AAV piggyBac transposon polynucleotide of any one of the preceding embodiments, wherein the at least two promoter sequences are different sequences.

Embodiment 44a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 7;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

Embodiment 44b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 7;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence.

Embodiment 44c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 7;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second nucleic acid sequence comprising the sequence of SEQ ID NO: 2.

Embodiment 44d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 7;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 80;
i) a second AAV ITR sequence.

Embodiment 45a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 8;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

Embodiment 45b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 8;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence.

Embodiment 45c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 8;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second nucleic acid sequence comprising the nucleic acid sequence of SEQ ID NO: 2.

Embodiment 45d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 8;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 80;
i) a second AAV ITR sequence.

Embodiment 46a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 9;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

Embodiment 46b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 9;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence.

Embodiment 46c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 9;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second nucleic acid sequence comprising the nucleic acid sequence of SEQ ID NO: 2.

Embodiment 46d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 9;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 80;
i) a second AAV ITR sequence.

Embodiment 47a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

Embodiment 47b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence.

Embodiment 47c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second nucleic acid sequence comprising the nucleic acid sequence of SEQ ID NO: 2.

Embodiment 47d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 80;
i) a second AAV ITR sequence.

Embodiment 48a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

Embodiment 48b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence.

Embodiment 48c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second nucleic sequence comprising the nucleic acid sequence of SEQ ID NO: 2.

Embodiment 48d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 80;
i) a second AAV ITR sequence.

Embodiment 49a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence comprising the nucleic acid sequence of SEQ ID NO: 2.

Embodiment 49b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence.

Embodiment 49c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second nucleic acid sequence comprising the nucleic acid sequence of SEQ ID NO: 2.

Embodiment 49d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 80; and
k) a second AAV ITR sequence.

Embodiment 50a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence comprising the nucleic acid sequence of SEQ ID NO: 2.

Embodiment 50b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence.

Embodiment 50c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second nucleic acid sequence comprising the nucleic acid sequence of SEQ ID NO: 2.

Embodiment 50d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 80; and
k) a second AAV ITR sequence.

Embodiment 51a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

Embodiment 51b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence.

Embodiment 51c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second nucleic acid sequence comprising the nucleic acid sequence of SEQ ID NO: 2.

Embodiment 51d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 80;
i) a second AAV ITR sequence.

Embodiment 52a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

Embodiment 52b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence.

Embodiment 52c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second nucleic acid sequence comprising the nucleic acid sequence of SEQ ID NO: 2.

Embodiment 52d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 80;
i) a second AAV ITR sequence.

Embodiment 53a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence comprising the nucleic acid sequence of SEQ ID NO: 2.

Embodiment 53b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence.

Embodiment 53c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second nucleic acid sequence comprising the nucleic acid sequence of SEQ ID NO: 2.

Embodiment 53d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 80; and
k) a second AAV ITR sequence.

Embodiment 54a. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence comprising the nucleic acid sequence of SEQ ID NO: 2.

Embodiment 54b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence.

Embodiment 54c. An AAV piggyBac transposon polynucleotide comprising in the 5′ to 3′ direction:
a) a first nucleic acid sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second nucleic acid sequence comprising the nucleic acid sequence of SEQ ID NO: 2.

Embodiment 54d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 80; and
k) a second AAV ITR sequence.

Embodiment 55a. An AAV piggyBac transposon polynucleotide comprising in the 5′ to 3′ direction:
a) a first AAV ITR sequence comprising the nucleic acid sequence of SEQ ID NO: 17;
b) a first piggyBac ITR sequence comprising the nucleic acid sequence of SEQ ID NO: 19;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) a polyA sequence comprising the nucleic acid sequence of SEQ ID NO: 22;
g) a second insulator sequence comprising the nucleic acid sequence of SEQ ID NO: 20;
h) a second piggyBac ITR sequence comprising the nucleic acid sequence of SEQ ID NO: 21;
i) a DNA spacer sequence comprising the nucleic acid sequence SEQ ID NO: 28
j) a second AAV ITR sequence comprising the nucleic acid sequence of SEQ ID NO: 18.

Embodiment 55b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 19;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) a polyA sequence comprising the nucleic acid sequence of SEQ ID NO: 22;
g) a second insulator sequence comprising the nucleic acid sequence of SEQ ID NO: 20;
h) a second piggyBac ITR sequence comprising the nucleic acid sequence of SEQ ID NO: 21;
i) a DNA spacer sequence comprising the nucleic acid sequence SEQ ID NO: 28
j) a second AAV ITR sequence.

Embodiment 55c. An AAV piggyBac transposon polynucleotide comprising in the 5′ to 3′ direction:
a) a first nucleic acid sequence comprising the nucleic acid sequence of SEQ ID NO: 17;
b) a first piggyBac ITR sequence comprising the nucleic acid sequence of SEQ ID NO: 19;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) a polyA sequence comprising the nucleic acid sequence of SEQ ID NO: 22;
g) a second insulator sequence comprising the nucleic acid sequence of SEQ ID NO: 20;
h) a second piggyBac ITR sequence comprising the nucleic acid sequence of SEQ ID NO: 21;
i) a DNA spacer sequence comprising the nucleic acid sequence SEQ ID NO: 28
j) a second nucleic acid sequence comprising the nucleic acid sequence of SEQ ID NO: 18.

Embodiment 55d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) a polyA sequence comprising the nucleic acid sequence of SEQ ID NO: 22;
g) a second insulator sequence comprising the nucleic acid sequence of SEQ ID NO: 20;
h) a second piggyBac ITR sequence comprising the nucleic acid sequence of SEQ ID NO: 80;
i) a DNA spacer sequence comprising the nucleic acid sequence SEQ ID NO: 28
j) a second AAV ITR sequence.

Embodiment 56a. An AAV piggyBac transposon polynucleotide comprising in the 5′ to 3′ direction:
a) a first AAV ITR sequence comprising the nucleic acid sequence of SEQ ID NO: 17;
b) a first piggyBac ITR sequence comprising the nucleic acid sequence of SEQ ID NO: 19;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 24;
g) an at least second transgene sequence comprising the nucleic acid sequence of SEQ ID NO: 25;
h) a polyA sequence comprising the nucleic acid sequence of SEQ ID NO: 22;
i) a second insulator sequence comprising the nucleic acid sequence of SEQ ID NO: 20;
j) a second piggyBac ITR sequence comprising the nucleic acid sequence of SEQ ID NO: 21; and
k) a second AAV ITR sequence comprising the nucleic acid sequence of SEQ ID NO: 18.

Embodiment 56b. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 19;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 24;
g) an at least second transgene sequence comprising the nucleic acid sequence of SEQ ID NO: 25;
h) a polyA sequence comprising the nucleic acid sequence of SEQ ID NO: 22;
i) a second insulator sequence comprising the nucleic acid sequence of SEQ ID NO: 20;
j) a second piggyBac ITR sequence comprising the nucleic acid sequence of SEQ ID NO: 21; and
k) a second AAV ITR sequence.

Embodiment 56c. An AAV piggyBac transposon polynucleotide comprising in the 5′ to 3′ direction:
a) a first nucleic acid sequence comprising the nucleic acid sequence of SEQ ID NO: 17;
b) a first piggyBac ITR sequence comprising the nucleic acid sequence of SEQ ID NO: 19;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 24;
g) an at least second transgene sequence comprising the nucleic acid sequence of SEQ ID NO: 25;
h) a polyA sequence comprising the nucleic acid sequence of SEQ ID NO: 22;
i) a second insulator sequence comprising the nucleic acid sequence of SEQ ID NO: 20;
j) a second piggyBac ITR sequence comprising the nucleic acid sequence of SEQ ID NO: 21; and
k) a second nucleic acid sequence comprising the nucleic acid sequence of SEQ ID NO: 18.

Embodiment 56d. An AAV piggyBac transposon polynucleotide comprising in the 5′ to 3′ direction:
a) a first AAV ITR sequence;
b) a first piggyBac ITR sequence comprising the nucleic acid sequence of SEQ ID NO: 79;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 24;
g) an at least second transgene sequence comprising the nucleic acid sequence of SEQ ID NO: 25;
h) a polyA sequence comprising the nucleic acid sequence of SEQ ID NO: 22;
i) a second insulator sequence comprising the nucleic acid sequence of SEQ ID NO: 20;
j) a second piggyBac ITR sequence comprising the nucleic acid sequence of SEQ ID NO: 80; and
k) a second AAV ITR sequence.

Embodiment 57. The AAV piggyBac transposon polynucleotide of any one of the preceding embodiments, wherein the AAV piggyBac transposon polynucleotide comprises the nucleic acid sequence of SEQ ID NO: 29.

Embodiment 58. The AAV piggyBac transposon polynucleotide of any one of the preceding embodiments, wherein the AAV piggyBac transposon polynucleotide comprises the nucleic acid sequence of SEQ ID NO: 30.

Embodiment 59. The AAV piggyBac transposon polynucleotide of any one of the preceding embodiments, wherein the AAV piggyBac transposon polynucleotide comprises the nucleic acid sequence of SEQ ID NO: 31.

Embodiment 60. The AAV piggyBac transposon polynucleotide of any one of the preceding embodiments, wherein the AAV piggyBac transposon polynucleotide comprises the nucleic acid sequence of SEQ ID NO: 32.

Embodiment 61. The AAV piggyBac transposon polynucleotide of any one of the preceding embodiments, wherein the AAV piggyBac transposon polynucleotide comprises the nucleic acid sequence of SEQ ID NO: 33.

Embodiment 62. The AAV piggyBac transposon polynucleotide of any one of the preceding embodiments, wherein the AAV piggyBac transposon polynucleotide comprises the nucleic acid sequence of SEQ ID NO: 34.

Embodiment 63. The AAV piggyBac transposon polynucleotide of any one of the preceding embodiments, wherein the AAV piggyBac transposon polynucleotide comprises the nucleic acid sequence of SEQ ID NO: 35.

Embodiment 64. The AAV piggyBac transposon polynucleotide of any one of the preceding embodiments, wherein the AAV piggyBac transposon polynucleotide comprises the nucleic acid sequence of SEQ ID NO: 36.

Embodiment 65. The AAV piggyBac transposon polynucleotide of any one of the preceding embodiments, wherein the AAV piggyBac transposon polynucleotide comprises the nucleic acid sequence of SEQ ID NO: 37.

Embodiment 66. The AAV piggyBac transposon polynucleotide of any one of the preceding embodiments, wherein the AAV piggyBac transposon polynucleotide comprises the nucleic acid sequence of SEQ ID NO: 38.

Embodiment 67. The AAV piggyBac transposon polynucleotide of any one of the preceding embodiments, wherein the AAV piggyBac transposon polynucleotide comprises the nucleic acid sequence of SEQ ID NO: 39.

Embodiment 68. The AAV piggyBac transposon polynucleotide of any one of the preceding embodiments, wherein the AAV piggyBac transposon polynucleotide comprises the nucleic acid sequence of SEQ ID NO: 40.

Embodiment 69. The AAV piggyBac transposon polynucleotide of any one of the preceding embodiments, wherein the AAV piggyBac transposon polynucleotide comprises the nucleic acid sequence of SEQ ID NO: 41.

Embodiment 70. A vector comprising the AAV piggyBac transposon polynucleotide of any one of the preceding embodiments.

Embodiment 71. The vector of any one of the preceding embodiments, wherein the vector is a viral vector.

Embodiment 72. The vector of any one of the preceding embodiments, wherein the viral vector is an adeno-associated virus (AAV) viral vector.

Embodiment 73. The vector of any one of the preceding embodiments, wherein the AAV viral vector is an AAV1, AAV2, AAV3, AAV4, AAV5, AAV6, AAV7, AAV8, AAV9, AAV10 or AAV 11 viral vector.

Embodiment 74. The vector of any one of the preceding embodiments, wherein the AAV viral vector is an AAV-KP-1 or AAV-NP59 viral vector, preferably wherein the AAV viral vector is an AAV-KP-1 viral vector.

Embodiment 75. A composition comprising the vector of any one of embodiments 70-74.

Embodiment 76. An AAV transposase polynucleotide comprising in the 5′ to 3′ direction a first AAV ITR sequence, at least one promoter sequence, at least one transposase sequence, a polyA sequence and a second AAV ITR sequence.

Embodiment 77. An AAV transposase polynucleotide comprising in the 5′ to 3′ direction:
a) a first AAV ITR sequence comprising the nucleic acid sequence of SEQ ID NO: 73;
b) at least one promoter sequence at least one promoter sequence comprising the nucleic acid sequence of 74;
c) at least one transposase sequence comprising the nucleic acid sequence of SEQ ID NO: 53;
d) a polyA sequence comprising the nucleic acid sequences of SEQ ID NO: 75;
e) at least one DNA spacer sequence comprising the nucleic acid sequences of SEQ ID NO: 76; and
f) a second AAV ITR sequence comprising the nucleic acid sequences of SEQ ID NO: 77.

Embodiment 78. The AAV piggyBac transposase polynucleotide of any one of the proceeding Embodiments, wherein the at least one promoter sequence comprises the nucleic acid sequence of SEQ ID NO: 78.

Embodiment 79. A vector comprising the AAV transposase polynucleotide of any one of the preceding embodiments.

Embodiment 80. The vector of any one of the preceding embodiments, wherein the vector is a viral vector.

Embodiment 81. The vector of any one of the preceding embodiments, wherein the viral vector is an adeno-associated virus (AAV) viral vector.

Embodiment 82. The vector of any one of the preceding embodiments, wherein the AAV viral vector is an AAV1, AAV2, AAV3, AAV4, AAV5, AAV6, AAV7, AAV8, AAV9, AAV10 or AAV 11 viral vector.

Embodiment 83. The vector of any one of the preceding embodiments, wherein the AAV viral vector is an AAV-KP-1 or AAV-NP59 AAV viral vector, preferably wherein the AAV viral vector is an AAV-KP-1 viral vector.

Embodiment 84. A composition comprising the vector of any one of embodiments 79-83.

Embodiment 85. A composition comprising the vector of any one of embodiments 70-74 and the vector of any one of embodiments 79-83.

Embodiment 86. A method of treating hemophilia in a subject in need thereof comprising administering to the subject at least one therapeutically effective amount of at least one polynucleotide, vector or composition of any one of the preceding embodiments.

Embodiment 87. A method of treating hemophilia in a subject in need thereof comprising administering to a subject:
a) at least one therapeutically effective amount of the AAV piggyBac transposon polynucleotide of any one of the preceding embodiments, or any one of the vectors and/or compositions of the preceding embodiments that comprise an AAV piggyBac transposon polynucleotide; and
b) at least one therapeutically effective amount of the AAV piggyBac transposase polynucleotide of any one of the preceding embodiments, or any one of the vectors and/or compositions of the preceding embodiments that comprise an AAV piggyBac transposase polynucleotide.

Embodiment 88. The method of embodiment 86 or 87, wherein the hemophilia is hemophilia A, hemophilia B, hemophilia C, parahemophila or any combination thereof.

Embodiment 89. The method of embodiment 88, wherein the hemophilia is hemophilia A.

Embodiment 90. The method of embodiment 88, wherein the hemophilia is hemophilia B.

Embodiment 91. A polynucleotide comprising in the 5′ to 3′ direction:
a) a first piggyBac ITR sequence;
b) a first insulator sequence;
c) at least one promoter sequence;
d) at least one transgene sequence;
e) a polyA sequence;
f) a second insulator sequence;
g) a second piggyBac ITR sequence.

Embodiment 92. The polynucleotide of embodiment 91, wherein the polynucleotide comprises DNA, cDNA, gDNA, RNA, mRNA or any combination thereof.

Embodiment 93. The polynucleotide of any one of the preceding embodiments, wherein the first and/or the second nucleic acid sequence comprises the nucleic acid sequence of any one of SEQ ID NOs: 1, 2, 17 and 18.

Embodiment 94. The polynucleotide of any one of the preceding embodiments, wherein the first piggyBac ITR sequence and/or the second piggyBac ITR sequence comprises the nucleic acid sequence of any one of SEQ ID NOs: 3, 4, 19 and 21.

Embodiment 95. The Polynucleotide of any one of the preceding embodiments, wherein the first piggyBac ITR sequence and/or the second piggyBac ITR sequence comprises the nucleic acid sequence of any one of SEQ ID NOs: 1 and 2.

Embodiment 96. The polynucleotide of any one of the preceding embodiments, wherein the first piggyBac ITR sequence and/or the second piggyBac ITR sequence comprises the nucleic acid sequence of any one of SEQ ID NOs: 79 and 80.

Embodiment 97. The polynucleotide of any one of the preceding embodiments, wherein the first piggyBac ITR sequence comprises the nucleic acid sequence of SEQ ID NO: 3 and the second piggyBac ITR comprises the nucleic acid sequence of SEQ ID NO: 4.

Embodiment 98. The polynucleotide of any one of the preceding embodiments, wherein the first piggyBac ITR sequence comprises the nucleic acid sequence of SEQ ID NO: 19 and the second piggyBac ITR comprises the nucleic acid sequence of SEQ ID NO: 21.

Embodiment 99. The polynucleotide of any one of the preceding embodiments, wherein the first piggyBac ITR sequence comprises the nucleic acid sequence of SEQ ID NO: 79 and the second piggyBac ITR comprises the nucleic acid sequence of SEQ ID NO: 80.

Embodiment 100. The polynucleotide of any one of the preceding embodiments, wherein the first insulator sequence and/or the second insulator sequence comprises the nucleic acid sequence of any one of SEQ ID NOs: 5, 6 and 20.

Embodiment 101. The polynucleotide of any one of the preceding embodiments, wherein the first insulator sequence comprises the nucleic acid sequence of SEQ ID NO: 5 and the second insulator sequence comprises the nucleic acid sequence of SEQ ID NO: 6.

Embodiment 102. The polynucleotide of any one of the preceding embodiments, wherein the first insulator sequence comprises the nucleic acid sequence of SEQ ID NO: 5 and the second insulator sequence comprises the nucleic acid sequence of SEQ ID NO: 20.

Embodiment 103. The polynucleotide of any one of the preceding embodiments, wherein the at least one promoter sequence comprises the nucleic acid sequence of any one of SEQ IDS NOs: 7-12.

Embodiment 104. The polynucleotide of any one of the preceding embodiments, wherein the at least one promoter sequence comprises the nucleic acid sequence of SEQ IDS NO: 7.

Embodiment 105. The polynucleotide of any one of the preceding embodiments, wherein the at least one promoter sequence comprises the nucleic acid sequence of SEQ IDS NO: 8.

Embodiment 106. The polynucleotide of any one of the preceding embodiments, wherein the at least one promoter sequence comprises the nucleic acid sequence of SEQ IDS NO: 9.

Embodiment 107. The polynucleotide of any one of the preceding embodiments, wherein the at least one promoter sequence comprises the nucleic acid sequence of SEQ IDS NO: 10.

Embodiment 108. The polynucleotide of any one of the preceding embodiments, wherein the at least one promoter sequence comprises the nucleic acid sequence of SEQ IDS NO: 11.

Embodiment 109. The polynucleotide of any one of the preceding embodiments, wherein the at least one promoter sequence comprises the nucleic acid sequence of SEQ IDS NO: 12.

Embodiment 110. The polynucleotide of any one of the preceding embodiments, wherein the at least one transgene sequence comprises a nucleic acid sequence encoding a Factor VIII (FVIII) polypeptide.

Embodiment 111. The polynucleotide of any one of the preceding embodiments, wherein the FVIII polypeptide comprises the amino acid sequence of SEQ ID NO: 26.

Embodiment 112. The polynucleotide of any one of the preceding embodiments, wherein the nucleic acid sequence encoding a FVIII polypeptide comprises the nucleic acid sequence of SEQ ID NO: 13.

Embodiment 113. The polynucleotide of any one of the preceding embodiments, wherein the at least one transgene sequence comprises a nucleic acid sequence encoding a Factor IX (FIX) polypeptide.

Embodiment 114. The polynucleotide of any one of the preceding embodiments, wherein the FIX polypeptide comprises the amino acid sequence of SEQ ID NO: 27.

Embodiment 115. The polynucleotide of any one of the preceding embodiments, wherein the nucleic acid sequence encoding a FIX polypeptide comprises the nucleic acid sequence of SEQ ID NO: 23.

Embodiment 116. The polynucleotide of any one of the preceding embodiments, wherein the at least one transgene sequence is operatively linked to the at least one promoter sequence.

Embodiment 117. The polynucleotide of any one of the preceding embodiments, wherein the expression of the at least one transgene sequence is controlled by the at least one promoter sequence.

Embodiment 118. The polynucleotide of any one of the preceding embodiments, wherein the polyA sequence comprises the nucleic acid sequence of SEQ ID NO: 14.

Embodiment 119. The polynucleotide of any one of the preceding embodiments, wherein the polyA sequence comprises the nucleic acid sequence of SEQ ID NO: 22.

Embodiment 120. The polynucleotide of any one of the preceding embodiments, wherein the polynucleotide further comprises at least a second transgene sequence.

Embodiment 121. The polynucleotide of any one of the preceding embodiments, wherein the at least second transgene sequence comprises a luciferase sequence.

Embodiment 122. The polynucleotide of any one of the preceding embodiments, wherein the luciferase sequence comprises SEQ ID NO: 16 or 25.

Embodiment 123. The polynucleotide of any one of the preceding embodiments, wherein the polynucleotide further comprises at least a second promoter sequence.

Embodiment 124. The polynucleotide of any one of the preceding embodiments, wherein the at least second promoter sequence is located between the at least one transgene sequence and the at least second transgene sequence.

Embodiment 125. The polynucleotide of any one of the preceding embodiments, wherein the polynucleotide further comprises at least one self-cleaving peptide sequence, wherein the at least one self-cleaving peptide sequence is a nucleic acid sequence encoding for a T2A peptide, GSG-T2A peptide, an E2A peptide, a GSG-E2A peptide, an F2A peptide, a GSG-F2A peptide, a P2A peptide, or a GSG-P2A peptide.

Embodiment 126. The polynucleotide of any one of the preceding embodiments, wherein the at least one self-cleaving peptide sequence is located between the at least one transgene sequence and the at least second transgene sequence.

Embodiment 127. The polynucleotide of any one of the preceding embodiments, wherein the polynucleotide comprises at least two transgene sequences.

Embodiment 128. The polynucleotide of any one of the preceding embodiments, wherein the at least two transgene sequences are the same sequence.

Embodiment 129. The polynucleotide of any one of the preceding embodiments, wherein the at least two transgene sequences are different sequences.

Embodiment 130. The polynucleotide of any one of the preceding embodiments, further comprising at least one DNA spacer sequence.

Embodiment 131. The polynucleotide of any one of the preceding embodiments, wherein the at least one DNA spacer sequence comprises the nucleic acid sequence of SEQ ID NO: 28.

Embodiment 132. The polynucleotide of any one of the preceding embodiments, wherein the polynucleotide comprises at least two promoter sequences.

Embodiment 133. The polynucleotide of any one of the preceding embodiments, wherein the at least two promoter sequences are the same sequence.

Embodiment 134. The polynucleotide of any one of the preceding embodiments, wherein the at least two promoter sequences are different sequences.

Embodiment 135. An polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 7;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 4.

Embodiment 136. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 7;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 80.

Embodiment 137. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 8;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 4.

Embodiment 138. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 8;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 80.

Embodiment 139. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 9;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 4.

Embodiment 140. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 9;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 80.

Embodiment 141. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 10;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 4.

Embodiment 142. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 10;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 80.

Embodiment 143. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 11;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 4.

Embodiment 144. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 11;
d) at least one transgene sequence comprising the nucleic acid sequence of SEQ ID NO: 13;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 80.

Embodiment 145. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 10;
d) a first transgene sequence comprising the nucleic acid sequence of SEQ ID NO: 13;
e) at least one self-cleaving peptide sequence comprising the nucleic acid sequence of SEQ ID NO: 15;
f) an at least second transgene sequence comprising the nucleic acid sequence of SEQ ID NO: 16;
g) a polyA sequence comprising the nucleic acid sequence of SEQ ID NO: 14;
h) a second insulator sequence comprising the nucleic acid sequence of SEQ ID NO: 6;
i) a second piggyBac ITR sequence comprising the nucleic acid sequence of SEQ ID NO: 4.

Embodiment 146. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 10;
d) a first transgene sequence comprising the nucleic acid sequence of SEQ ID NO: 13;
e) at least one self-cleaving peptide sequence comprising the nucleic acid sequence of SEQ ID NO: 15;
f) an at least second transgene sequence comprising the nucleic acid sequence of SEQ ID NO: 16;
g) a polyA sequence comprising the nucleic acid sequence of SEQ ID NO: 14;
h) a second insulator sequence comprising the nucleic acid sequence of SEQ ID NO: 6;
i) a second piggyBac ITR sequence comprising the nucleic acid sequence of SEQ ID NO: 80.

Embodiment 147. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 12;
d) a first transgene sequence comprising the nucleic acid sequence of SEQ ID NO: 13;
e) at least one self-cleaving peptide sequence comprising the nucleic acid sequence of SEQ ID NO: 15;
f) an at least second transgene sequence comprising the nucleic acid sequence of SEQ ID NO: 16;
g) a polyA sequence comprising the nucleic acid sequence of SEQ ID NO: 14;
h) a second insulator sequence comprising the nucleic acid sequence of SEQ ID NO: 6;
i) a second piggyBac ITR sequence comprising the nucleic acid sequence of SEQ ID NO: 4.

Embodiment 148. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 12;
d) a first transgene sequence comprising the nucleic acid sequence of SEQ ID NO: 13;
e) at least one self-cleaving peptide sequence comprising the nucleic acid sequence of SEQ ID NO: 15;
f) an at least second transgene sequence comprising the nucleic acid sequence of SEQ ID NO: 16;
g) a polyA sequence comprising the nucleic acid sequence of SEQ ID NO: 14;
h) a second insulator sequence comprising the nucleic acid sequence of SEQ ID NO: 6;
i) a second piggyBac ITR sequence comprising the nucleic acid sequence of SEQ ID NO: 80.

Embodiment 149. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 11;
d) at least one transgene sequence comprising the nucleic acid sequence of SEQ ID NO: 23;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 4.

Embodiment 150. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 11;
d) at least one transgene sequence comprising the nucleic acid sequence of SEQ ID NO: 23;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 80.

Embodiment 151. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 10;
d) at least one transgene sequence comprising the nucleic acid sequence of SEQ ID NO: 23;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 4.

Embodiment 152. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 10;
d) at least one transgene sequence comprising the nucleic acid sequence of SEQ ID NO: 23;
e) a polyA sequence comprising the nucleic acid sequence of SEQ ID NO: 14;
f) a second insulator sequence comprising the nucleic acid sequence of SEQ ID NO: 6;
g) a second piggyBac ITR sequence comprising the nucleic acid sequence of SEQ ID NO: 80.

Embodiment 153. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 10;
d) a first transgene sequence comprising the nucleic acid sequence of SEQ ID NO: 23;
e) at least one self-cleaving peptide sequence comprising the nucleic acid sequence of SEQ ID NO: 15;
f) an at least second transgene sequence comprising the nucleic acid sequence of SEQ ID NO: 16;
g) a polyA sequence comprising the nucleic acid sequence of SEQ ID NO: 14;
h) a second insulator sequence comprising the nucleic acid sequence of SEQ ID NO: 6;
i) a second piggyBac ITR sequence comprising the nucleic acid sequence of SEQ ID NO: 4.

Embodiment 154. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 10;
d) a first transgene sequence comprising the nucleic acid sequence of SEQ ID NO: 23;
e) at least one self-cleaving peptide sequence comprising the nucleic acid sequence of SEQ ID NO: 15;
f) an at least second transgene sequence comprising the nucleic acid sequence of SEQ ID NO: 16;
g) a polyA sequence comprising the nucleic acid sequence of SEQ ID NO: 14;
h) a second insulator sequence comprising the nucleic acid sequence of SEQ ID NO: 6;
i) a second piggyBac ITR sequence comprising the nucleic acid sequence of SEQ ID NO: 80.

Embodiment 155. A polynucleotide comprising in the 5′ to 3′ direction:
a) a first piggyBac ITR sequence comprising the nucleic acid sequence of SEQ ID NO: 3;
b) a first insulator sequence comprising the nucleic acid sequence of SEQ ID NO: 5;
c) at least one promoter sequence comprising the nucleic acid sequence of SEQ ID NO: 12;
d) a first transgene sequence comprising the nucleic acid sequence of SEQ ID NO: 23;
e) at least one self-cleaving peptide sequence comprising the nucleic acid sequence of SEQ ID NO: 15;
f) an at least second transgene sequence comprising the nucleic acid sequence of SEQ ID NO: 16;
g) a polyA sequence comprising the nucleic acid sequence of SEQ ID NO: 14;
h) a second insulator sequence comprising the nucleic acid sequence of SEQ ID NO: 6;
i) a second piggyBac ITR sequence comprising the nucleic acid sequence of SEQ ID NO: 4.

Embodiment 156. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 12;
d) a first transgene sequence comprising the nucleic acid sequence of SEQ ID NO: 23;
e) at least one self-cleaving peptide sequence comprising the nucleic acid sequence of SEQ ID NO: 15;
f) an at least second transgene sequence comprising the nucleic acid sequence of SEQ ID NO: 16;
g) a polyA sequence comprising the nucleic acid sequence of SEQ ID NO: 14;
h) a second insulator sequence comprising the nucleic acid sequence of SEQ ID NO: 6;
i) a second piggyBac ITR sequence comprising the nucleic acid sequence of SEQ ID NO: 80.

Embodiment 157. A polynucleotide comprising in the 5′ to 3′ direction:
a) a first piggyBac ITR sequence comprising the nucleic acid sequence of SEQ ID NO: 19;
b) a first insulator sequence comprising the nucleic acid sequence of SEQ ID NO: 5;
c) at least one promoter sequence comprising the nucleic acid sequence of SEQ ID NO: 12;
d) a first transgene sequence comprising the nucleic acid sequence of SEQ ID NO: 23;
e) a polyA sequence comprising the nucleic acid sequence of SEQ ID NO: 22;
f) a second insulator sequence comprising the nucleic acid sequence of SEQ ID NO: 20;
g) a second piggyBac ITR sequence comprising the nucleic acid sequence of SEQ ID NO: 21;
h) a DNA spacer sequence comprising the nucleic acid sequence SEQ ID NO: 28.

Embodiment 158. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 12;
d) a first transgene sequence comprising the nucleic acid sequence of SEQ ID NO: 23;
e) a polyA sequence comprising the nucleic acid sequence of SEQ ID NO: 22;
f) a second insulator sequence comprising the nucleic acid sequence of SEQ ID NO: 20;
g) a second piggyBac ITR sequence comprising the nucleic acid sequence of SEQ ID NO: 80;
h) a DNA spacer sequence comprising the nucleic acid sequence SEQ ID NO: 28.

Embodiment 159. A polynucleotide comprising in the 5′ to 3′ direction:
a) a first piggyBac ITR sequence comprising the nucleic acid sequence of SEQ ID NO: 19;
b) a first insulator sequence comprising the nucleic acid sequence of SEQ ID NO: 5;
c) at least one promoter sequence comprising the nucleic acid sequence of SEQ ID NO: 12;
d) a first transgene sequence comprising the nucleic acid sequence of SEQ ID NO: 23;
e) at least one self-cleaving peptide sequence comprising the nucleic acid sequence of SEQ ID NO: 24;
f) an at least second transgene sequence comprising the nucleic acid sequence of SEQ ID NO: 25;
g) a polyA sequence comprising the nucleic acid sequence of SEQ ID NO: 22;
h) a second insulator sequence comprising the nucleic acid sequence of SEQ ID NO: 20;
i) a second piggyBac ITR sequence comprising the nucleic acid sequence of SEQ ID NO: 21.

Embodiment 160. A polynucleotide comprising in the 5′ to 3′ direction:
a) a first piggyBac ITR sequence comprising the nucleic acid sequence of SEQ ID NO: 79;
b) a first insulator sequence comprising the nucleic acid sequence of SEQ ID NO: 5;
c) at least one promoter sequence comprising the nucleic acid sequence of SEQ ID NO: 12;
d) a first transgene sequence comprising the nucleic acid sequence of SEQ ID NO: 23;
e) at least one self-cleaving peptide sequence comprising the nucleic acid sequence of SEQ ID NO: 24;
f) an at least second transgene sequence comprising the nucleic acid sequence of SEQ ID NO: 25;
g) a polyA sequence comprising the nucleic acid sequence of SEQ ID NO: 22;
h) a second insulator sequence comprising the nucleic acid sequence of SEQ ID NO: 20;
i) a second piggyBac ITR sequence comprising the nucleic acid sequence of SEQ ID NO: 80.

Embodiment 161. The polynucleotide of any one of the preceding embodiments, wherein the polynucleotide comprises the nucleic acid sequence of SEQ ID NO: 29.

Embodiment 162. The polynucleotide of any one of the preceding embodiments, wherein the polynucleotide comprises the nucleic acid sequence of SEQ ID NO: 30.

Embodiment 163. The polynucleotide of any one of the preceding embodiments, wherein the polynucleotide comprises the nucleic acid sequence of SEQ ID NO: 31.

Embodiment 164. The polynucleotide of any one of the preceding embodiments, wherein the polynucleotide comprises the nucleic acid sequence of SEQ ID NO: 32.

Embodiment 165. The polynucleotide of any one of the preceding embodiments, wherein the polynucleotide comprises the nucleic acid sequence of SEQ ID NO: 33.

Embodiment 166. The polynucleotide of any one of the preceding embodiments, wherein the polynucleotide comprises the nucleic acid sequence of SEQ ID NO: 34.

Embodiment 167. The polynucleotide of any one of the preceding embodiments, wherein the polynucleotide comprises the nucleic acid sequence of SEQ ID NO: 35.

Embodiment 168. The polynucleotide of any one of the preceding embodiments, wherein the polynucleotide comprises the nucleic acid sequence of SEQ ID NO: 36.

Embodiment 169. The polynucleotide of any one of the preceding embodiments, wherein the polynucleotide comprises the nucleic acid sequence of SEQ ID NO: 37.

Embodiment 170. The polynucleotide of any one of the preceding embodiments, wherein the polynucleotide comprises the nucleic acid sequence of SEQ ID NO: 38.

Embodiment 171. The polynucleotide of any one of the preceding embodiments, wherein the polynucleotide comprises the nucleic acid sequence of SEQ ID NO: 39.

Embodiment 172. The polynucleotide of any one of the preceding embodiments, wherein the polynucleotide comprises the nucleic acid sequence of SEQ ID NO: 40.

Embodiment 173. The polynucleotide of any one of the preceding embodiments, wherein the polynucleotide comprises the nucleic acid sequence of SEQ ID NO: 41.

Embodiment 174. A vector comprising the polynucleotide of any one of the preceding embodiments.

Embodiment 175. The vector of any one of the preceding embodiments, wherein the vector is a viral vector.

Embodiment 176. The vector of any one of the preceding embodiments, wherein the viral vector is an adeno-associated virus (AAV) viral vector.

Embodiment 177. The vector of any one of the preceding embodiments, wherein the AAV viral vector is an AAV1, AAV2, AAV3, AAV4, AAV5, AAV6, AAV7, AAV8, AAV9, AAV10 or AAV 11 viral vector.

Embodiment 178. The vector of any one of the preceding embodiments, wherein the AAV viral vector is an AAV-KP-1 or AAV-NP59 viral vector, preferably wherein the AAV viral vector is an AAV-KP-1 viral vector.

Embodiment 179. A composition comprising the vector or polynucleotide of any one of the preceding embodiments.

Embodiment 180. A method of treating hemophilia in a subject in need thereof comprising administering to the subject at least one therapeutically effective amount of at least one polynucleotide, vector or composition of any one of the preceding embodiments.

Embodiment 181. The method of embodiment 181, wherein the hemophilia is hemophilia A, hemophilia B, hemophilia C, parahemophila or any combination thereof.

Embodiment 182. The method of embodiment 182, wherein the hemophilia is hemophilia A.

Embodiment 183. The method of embodiment 182, wherein the hemophilia is hemophilia B.

### Definitions

### Nucleic Acid and Polynucleotide Molecules

Nucleic acid molecules and polynucleotide molecules of the present disclosure can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

### Construction of Nucleic Acid and Polynucleotide Molecules

The nucleic acid and polynucleotide molecules of the present disclosure can be made using (a) recombinant methods, (b) synthetic techniques, (c) purification techniques, and/or (d) combinations thereof, as well-known in the art.

The nucleic acid and polynucleotide molecules can conveniently comprise nucleotide sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art.

### Recombinant Methods for Constructing Nucleic Acid and Polynucleotide Molecules

The nucleic acid and polynucleotide molecules of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art.

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference.

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The nucleic acid and polynucleotide molecules of the disclosure can also be prepared by direct chemical synthesis by known methods. Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid or polynucleotide molecule of the present disclosure. A nucleic acid or polynucleotide of the present disclosure can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also relates to vectors that include isolated nucleic acid and polynucleotide molecules of the disclosure, host cells that are genetically engineered with the recombinant vectors, and the production of at least polynucleotide by recombinant techniques, as is well known in the art.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464; 5,770,359; 5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124(8):1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

Those of ordinary skill in the art are knowledgeable in the numerous expression systems available for expression of a nucleic acid or polynucleotide molecule. Alternatively, nucleic acids of the disclosure can be expressed in a host cell by turning on (by manipulation) in a host cell that contains endogenous DNA encoding a nucleic acid or polynucleotide of the present disclosure. Such methods are well known in the art, e.g., as described in U.S. Pat. Nos. 5,580,734, 5,641,670, 5,733,746, and 5,733,761, entirely incorporated herein by reference

Illustrative of cell cultures useful for the production of the nucleic acid and polynucleotide molecules of the present disclosure, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Agl4, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Agl4 cells (ATCC Accession Number CRL-1851). In a preferred aspect, the recombinant cell is a P3X63Ab8.653 or an SP2/0-Agl4 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof. In some aspects, a transgene sequence, or any other sequence, is said to be operatively linked to a promoter sequence when the promoter sequence controls the expression of the transgene sequence, or any other sequence. In some aspects, a transposase sequence is said to be operatively linked to a promoter sequence when the promoter sequence controls the expression of the transposase sequence.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof, (iii) a nucleic acid that is substantially identical to a referenced nucleic acid or the complement thereof, or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within 2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| | |
|---|---|
| Side chain characteristics | Amino Acid |

| | | |
|---|---|---|
| Aliphatic | Non-polar | G A P I L V F |
| | Polar - uncharged | C S T M N Q |
| | Polar - charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| | |
|---|---|
| Side Chain Characteristic | Amino Acid |

| | | |
|---|---|---|
| Non-polar (hydrophobic) | Aliphatic: | A L I V P |
| | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

The terms “effective amount” and “therapeutically effective amount” of an agent or compound are used in the broadest sense to refer to a nontoxic but sufficient amount of an active agent or compound to provide the desired effect or benefit. It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

### Example 1 AAV Viral Vectors of the Present Disclosure for the Treatment of Hemophilia

The following is a non-limiting example demonstrating the compositions and methods of the present disclosure can be used to in the treatment of hemophilia, and more specifically hemophilia B.

Three-week old C57BL/6 mice were either left untreated, or were administered one of the following two treatments:
Treatment #1: AAV piggyBac transposon vectors of the present disclosure at 3.75×10¹³ vg/kg
Treatment #2: AAV piggyBac transposon vectors of the present disclosure at 2.5×10¹³ vg/kg in combination with AAV transposase vectors of the present disclosure at 1.25×10¹³ vg/kg.

The AAV piggyBac transposon vectors of the present disclosure were AAV viral vector particles comprising an AAV piggyBac transposon polynucleotide of the present disclosure comprising a TTRm promoter sequence and a transgene sequence encoding for a Factor IX polypeptide. The AAV viral vector particles were KP-1 AAV viral vector particles.

The AAV transposase vector of the present disclosure were AAV viral vector particles comprising an AAV transposase polynucleotide of the present disclosure comprising an HLP promoter sequence and a transposase sequence encoding for a Super piggyBac transposase. The AAV viral vector particles were KP-1 AAV viral vector particles.

Three weeks following administration of the treatments, ELISA experiments were performed to determine the amount of human Factor IX polypeptide in the plasma of the mice.

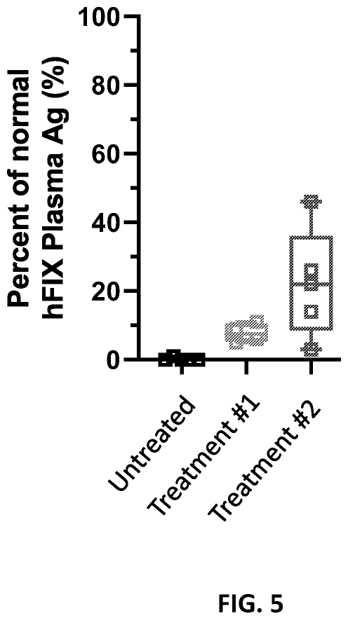

The results of these ELISA experiments are shown in FIG. 5. FIG. 5 shows that human Factor IX protein levels were observed in the samples at a level corresponding to between about 10-35% of normal human Factor IX following administration of Treatment #2.

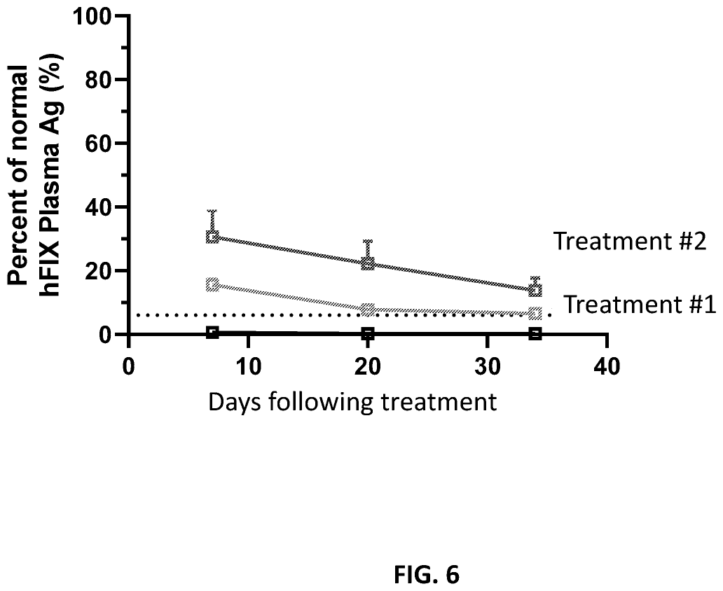

Additionally, similar ELISA experiments were performed at various time points following administration of the treatment. The results of these ELISA experiments are shown in FIG. 6.

The results presented in this example demonstrate that the AAV piggyBac transposon vectors and AAV transposase vectors of the present disclosure can be used to drive high levels of Factor IX expression in vivo, thereby demonstrating that the composition and methods of the present disclosure can be used to treat hemophilia B.

### EQUIVALENTS

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. An AAV piggyBac transposon polynucleotide comprising in the 5′ to 3′ direction:
a) a first AAV ITR sequence comprising the nucleic acid sequence of SEQ ID NO: 17;
b) a first piggyBac ITR sequence comprising the nucleic acid sequence of SEQ ID NO: 19;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) a polyA sequence comprising the nucleic acid sequence of SEQ ID NO: 22;
g) a second insulator sequence comprising the nucleic acid sequence of SEQ ID NO: 20;
h) a second piggyBac ITR sequence comprising the nucleic acid sequence of SEQ ID NO: 21;
i) a DNA spacer sequence comprising the nucleic acid sequence SEQ ID NO: 28
j) a second AAV ITR sequence comprising the nucleic acid sequence of SEQ ID NO: 18.

2. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 7;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

3. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 8;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

4. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 9;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

5. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

6. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 13;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

7. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence comprising the nucleic acid sequence of SEQ ID NO: 2.

8. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 13;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence comprising the nucleic acid sequence of SEQ ID NO: 2.

9. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 11;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

10. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) at least one transgene sequence comprising the nucleic acid sequence of SEQ ID NO: 23;
f) a polyA sequence comprising the nucleic acid sequence of SEQ ID NO: 14;
g) a second insulator sequence comprising the nucleic acid sequence of SEQ ID NO: 6;
h) a second piggyBac ITR sequence comprising the nucleic acid sequence of SEQ ID NO: 4;
i) a second AAV ITR sequence comprising the nucleic acid sequence of SEQ ID NO: 2.

11. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 10;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence comprising the nucleic acid sequence of SEQ ID NO: 2.

12. An AAV piggyBac transposon polynucleotide comprising in the 5′ to 3′ direction:
a) a first AAV ITR sequence comprising the nucleic acid sequence of SEQ ID NO: 1;
b) a first piggyBac ITR sequence comprising the nucleic acid sequence of SEQ ID NO: 3;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 15;
g) an at least second transgene sequence comprising the nucleic acid sequence of SEQ ID NO: 16;
h) a polyA sequence comprising the nucleic acid sequence of SEQ ID NO: 14;
i) a second insulator sequence comprising the nucleic acid sequence of SEQ ID NO: 6;
j) a second piggyBac ITR sequence comprising the nucleic acid sequence of SEQ ID NO: 4; and
k) a second AAV ITR sequence comprising the nucleic acid sequence of SEQ ID NO: 2.

13. An AAV piggyBac transposon polynucleotide comprising in the 5′ to 3′ direction:
a) a first AAV ITR sequence comprising the nucleic acid sequence of SEQ ID NO: 17;
b) a first piggyBac ITR sequence comprising the nucleic acid sequence of SEQ ID NO: 19;
c) a first insulator sequence comprising the nucleic acid sequence of SEQ ID NO: 5;
d) at least one promoter sequence comprising the nucleic acid sequence of SEQ ID NO: 12;
e) a first transgene sequence comprising the nucleic acid sequence of SEQ ID NO: 23;
f) at least one self-cleaving peptide sequence comprising the nucleic acid sequence of SEQ ID NO: 24;
g) an at least second transgene sequence comprising the nucleic acid sequence of SEQ ID NO: 25;
h) a polyA sequence comprising the nucleic acid sequence of SEQ ID NO: 22;
i) a second insulator sequence comprising the nucleic acid sequence of SEQ ID NO: 20;
j) a second piggyBac ITR sequence comprising the nucleic acid sequence of SEQ ID NO: 21; and
k) a second AAV ITR sequence comprising the nucleic acid sequence of SEQ ID NO: 18.

14. The AAV piggyBac transposon polynucleotide of any one of the preceding claims, wherein the AAV piggyBac transposon polynucleotide comprises the nucleic acid sequence of SEQ ID NO: 29.

15. The AAV piggyBac transposon polynucleotide of any one of the preceding claims, wherein the AAV piggyBac transposon polynucleotide comprises the nucleic acid sequence of SEQ ID NO: 30.

16. The AAV piggyBac transposon polynucleotide of any one of the preceding claims, wherein the AAV piggyBac transposon polynucleotide comprises the nucleic acid sequence of SEQ ID NO: 31.

17. The AAV piggyBac transposon polynucleotide of any one of the preceding claims, wherein the AAV piggyBac transposon polynucleotide comprises the nucleic acid sequence of SEQ ID NO: 32.

18. The AAV piggyBac transposon polynucleotide of any one of the preceding claims, wherein the AAV piggyBac transposon polynucleotide comprises the nucleic acid sequence of SEQ ID NO: 33.

19. The AAV piggyBac transposon polynucleotide of any one of the preceding claims, wherein the AAV piggyBac transposon polynucleotide comprises the nucleic acid sequence of SEQ ID NO: 34.

20. The AAV piggyBac transposon polynucleotide of any one of the preceding claims, wherein the AAV piggyBac transposon polynucleotide comprises the nucleic acid sequence of SEQ ID NO: 35.

21. The AAV piggyBac transposon polynucleotide of any one of the preceding claims, wherein the AAV piggyBac transposon polynucleotide comprises the nucleic acid sequence of SEQ ID NO: 36.

22. The AAV piggyBac transposon polynucleotide of any one of the preceding claims, wherein the AAV piggyBac transposon polynucleotide comprises the nucleic acid sequence of SEQ ID NO: 37.

23. The AAV piggyBac transposon polynucleotide of any one of the preceding claims, wherein the AAV piggyBac transposon polynucleotide comprises the nucleic acid sequence of SEQ ID NO: 38.

24. The AAV piggyBac transposon polynucleotide of any one of the preceding claims, wherein the AAV piggyBac transposon polynucleotide comprises the nucleic acid sequence of SEQ ID NO: 39.

25. The AAV piggyBac transposon polynucleotide of any one of the preceding claims, wherein the AAV piggyBac transposon polynucleotide comprises the nucleic acid sequence of SEQ ID NO: 40.

26. The AAV piggyBac transposon polynucleotide of any one of the preceding claims, wherein the AAV piggyBac transposon polynucleotide comprises the nucleic acid sequence of SEQ ID NO: 41.

27. A vector comprising the AAV piggyBac transposon polynucleotide of any one of the preceding claims.

28. The vector of claim 27, wherein the vector is a viral vector, preferably wherein the viral vector is an AAV viral vector.

29. The vector of claim 28, wherein the AAV viral vector is an AAV1, AAV2, AAV3, AAV4, AAV5, AAV6, AAV7, AAV8, AAV9, AAV10 or AAV11 viral vector.

30. The vector of claim 28, wherein the AAV viral vector is an AAV-KP-1 or AAV-NP59 viral vector, preferably wherein the AAV viral vector is an AAV-KP-1 viral vector.

31. A composition comprising the vector of any one of claims 27-30.

32. An AAV transposase polynucleotide comprising in the 5′ to 3′ direction:
a) a first AAV ITR sequence comprising the nucleic acid sequence of SEQ ID NO: 73;
b) at least one promoter sequence at least one promoter sequence comprising the nucleic acid sequence of 74;
c) at least one transposase sequence comprising the nucleic acid sequence of SEQ ID NO: 53;
d) a polyA sequence comprising the nucleic acid sequences of SEQ ID NO: 75;
e) at least one DNA spacer sequence comprising the nucleic acid sequences of SEQ ID NO: 76; and
f) a second AAV ITR sequence comprising the nucleic acid sequences of SEQ ID NO: 77.

33. The AAV piggyBac transposase polynucleotide of any one of the proceeding claims, wherein the at least one promoter sequence comprises the nucleic acid sequence of SEQ ID NO: 78.

34. A vector comprising the AAV piggyBac transposon polynucleotide of claim 32 or claim 33.

35. The vector of claim 34, wherein the vector is a viral vector, preferably wherein the viral vector is an AAV viral vector.

36. The vector of claim 35, wherein the AAV viral vector is an AAV1, AAV2, AAV3, AAV4, AAV5, AAV6, AAV7, AAV8, AAV9, AAV10 or AAV11 viral vector.

37. The vector of claim 35, wherein the AAV viral vector is an AAV-KP-1 or AAV-NP59 viral vector, preferably wherein the AAV viral vector is an AAV-KP-1 viral vector.

38. A composition comprising the vector of any one of claims 34-37.

39. A method of treating a hemophilia in a subject in need thereof comprising administering to the subject at least one therapeutically effective dose of the polynucleotide, vector or composition of any one of the preceding claims.

40. A method of treating hemophilia in a subject in need thereof, the method comprising administering to the subject:
a) the polynucleotide of any one of claims 1-26, the vector of any one of claims 27-30 or the composition of claim 31; and
b) the polynucleotide of any one of claims 32-33, the vector of any one of claims 34-37 or the composition of claim 38.

41. The use of the polynucleotide, vector or composition of any one of the preceding claims for the treatment of hemophilia in a subject in need thereof, wherein the polynucleotide, vector or composition is for administration to the subject in at least one therapeutically effective amount.

42. The combination of
a) the polynucleotide of any one of claims 1-26, the vector of any one of claims 27-30 or the composition of claim 31; and
b) the polynucleotide of any one of claims 32-33, the vector of any one of claims 34-37 or the composition of claim 38
for use in the treatment of hemophilia in a subject in need thereof.

43. The method or use of any one of claims 39-42, wherein the hemophilia is hemophilia A, hemophilia B, hemophilia C, parahemophila or any combination thereof.

44. The method or use of claim 43, wherein the hemophilia is hemophilia A.

45. The method or use of claim 43, wherein the hemophilia is hemophilia B.


SEQUENCE LISTING (80 sequences)

SEQ ID NO: 1 - DNA, 63 bp, Artificial Sequence
  Synthetic Sequence
ccctagaaagataatcatattgtgacgtacgttaaagataatcatgcgtaaaattgacgcatg

SEQ ID NO: 2 - DNA, 35 bp, Artificial Sequence
  Synthetic Sequence
catgcgtcaattttacgcagactatctttctaggg

SEQ ID NO: 3 - DNA, 175 bp, Artificial Sequence
  Synthetic Sequence
tgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 4 - DNA, 274 bp, Artificial Sequence
  Synthetic Sequence
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatg

SEQ ID NO: 5 - DNA, 232 bp, Artificial Sequence
  Synthetic Sequence
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 6 - DNA, 231 bp, Artificial Sequence
  Synthetic Sequence
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccct

SEQ ID NO: 7 - DNA, 727 bp, Artificial Sequence
  Synthetic Sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 8 - DNA, 689 bp, Artificial Sequence
  Synthetic Sequence
cgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccacc

SEQ ID NO: 9 - DNA, 689 bp, Artificial Sequence
  Synthetic Sequence
cgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgctagc

SEQ ID NO: 10 - DNA, 252 bp, Artificial Sequence
  Synthetic Sequence
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 11 - DNA, 341 bp, Artificial Sequence
  Synthetic Sequence
gtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaatt

SEQ ID NO: 12 - DNA, 336 bp, Artificial Sequence
  Synthetic Sequence
gtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacag

SEQ ID NO: 13 - DNA, 4371 bp, Artificial Sequence
  Synthetic Sequence
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtac

SEQ ID NO: 14 - DNA, 192 bp, Artificial Sequence
  Synthetic Sequence
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 15 - DNA, 54 bp, Artificial Sequence
  Synthetic Sequence
gaaggacggggaagcctgcttacatgcggagatgtggaggagaatcctggtccc

SEQ ID NO: 16 - DNA, 513 bp, Artificial Sequence
  Synthetic Sequence
atggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcc

SEQ ID NO: 17 - DNA, 130 bp, Artificial Sequence
  Synthetic Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 18 - DNA, 141 bp, Artificial Sequence
  Synthetic Sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 19 - DNA, 213 bp, Artificial Sequence
  Synthetic Sequence
cgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 20 - DNA, 232 bp, Artificial Sequence
  Synthetic Sequence
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctc

SEQ ID NO: 21 - DNA, 309 bp, Artificial Sequence
  Synthetic Sequence
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 22 - DNA, 122 bp, Artificial Sequence
  Synthetic Sequence
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 23 - DNA, 1383 bp, Artificial Sequence
  Synthetic Sequence
atgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcacc

SEQ ID NO: 24 - DNA, 63 bp, Artificial Sequence
  Synthetic Sequence
ggatccggagaaggacggggaagcctgcttacatgcggagatgtggaggagaatcctggtccc

SEQ ID NO: 25 - DNA, 628 bp, Artificial Sequence
  Synthetic Sequence
atggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatga

SEQ ID NO: 26 - protein, 1457 aa, Artificial Sequence
  Synthetic Sequence
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 27 - protein, 461 aa, Artificial Sequence
  Synthetic Sequence
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 28 - DNA, 1045 bp, Artificial Sequence
  Synthetic Sequence
tttcctggactacttcagacgaacttcgtagggcgcataagtctcgaccacgcaatgacgcagcgatgcttgaaaaaaacaccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtcacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgc

SEQ ID NO: 29 - DNA, 6724 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattaaccaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccgcggtctagaaagaggtaagggtttaagggatggttggttggtggggtattaatgtttaattacctggagcacctgcctgaaatcactttttttcaggttggacgcgtcgctagcatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 30 - DNA, 6550 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 31 - DNA, 6550 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatgggaattgtaccgcgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccacggatcccactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggacgcgtcgctagcatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 32 - DNA, 6172 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattctaatacgacgtctctgacctaggccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 33 - DNA, 6234 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 34 - DNA, 6748 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattctaatacgacgtctctgacctaggccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtacggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 35 - DNA, 6810 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtacggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 36 - DNA, 3246 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcacctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 37 - DNA, 3184 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattctaatacgacgtctctgacctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcacctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 38 - DNA, 3760 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattctaatacgacgtctctgacctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcaccggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 39 - DNA, 3822 bp, Artificial Sequence
  Synthetic Sequence
ttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcaccggctccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgaaacgttagatctggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaa

SEQ ID NO: 40 - DNA, 4582 bp, Artificial Sequence
  Synthetic Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcaaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcacctaatgaagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgctttcctggactacttcagacgaacttcgtagggcgcataagtctcgaccacgcaatgacgcagcgatgcttgaaaaaaacaccgctttcaaggcggctggacgaagaccgcaagacaccctccacctcacctagcctgtatctctgcaatagcctaattacttcggaatctcctgtcgtaattccttagataaacggcaattaggtgacactctaaatctgtgtggaaccggcttccaaacactctaccacctctattagtgacacagagagaatccttgagtggcttctaggtatattgaacaacttcatcacgaattgagcagtgatcatggttctacgtatcaaccaatattaaccactgtgctctgtagcattgctaaatcgggctgtctgtttcaccatagatcgtgaggccatgcccacgggcattagaacttagcctgtttagcgataatcccaacaatgagctgggatatgacgagaagtatttagcaaccttttcgtgatcggctacgtaaaacctcatattacggcatgtacctgatcattgacctcaggccatacgcatgtgggagaatagagggaatagcacgatatactgcctcccttatcttcctcaggttgaggcgcaatgattatcactgctacgggcacagtgttactatcgggatcagggactgtgtctgtacccggttctaccacgccgtagctcccacattgtcgccgtcacgtcgttagggccagcctgcgcttggtacgtgatcgaggagtatcgtcttgtgcacacaaacaaaatagactgggtcccaagcgcacttagacaaggcattgtactactcaagtgttggatgtgaggaatgatacaatcgaaccccgtcgttcggtcaacaatttggctagagctgtgttgatacacagcggacgggagttacggggacagtcctccgagctggccagcaagatgcagctgacgcctgcaccggaagtaatccggaggccggccaggcctcctgcgagggggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 41 - DNA, 4137 bp, Artificial Sequence
  Synthetic Sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgaagactcttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaactcgtcgactaggcgcggtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggaattcctaggccaccatgcagcgcgtgaacatgattatggccgagtctcccggcctgatcaccatctgtctgctgggctatctgctgagcgccgagtgcaccgtgtttctggatcacgagaacgccaacaagatcctgaacagacccaagcggtacaacagcggcaaactggaagagttcgtgcagggtaacctggaacgcgagtgcatggaagagaagtgcagcttcgaagaggccagagaggtgttcgagaacaccgagagaaccaccgagttctggaagcagtacgtggacggcgatcagtgcgagagcaacccttgtctgaatggcggcagctgcaaggatgacatcaacagctacgagtgctggtgccccttcggcttcgagggcaagaattgcgagctggatgtgacctgcaacatcaagaacggcagatgcgagcagttctgcaagaacagcgccgacaacaaggtcgtgtgctcctgcacagagggctacagactggccgagaatcagaagtcctgcgagcccgctgtgccctttccatgtggcagagtgtctgtgtcccagaccagcaagctgaccagagccgagacagtgttccccgatgtggactacgtgaacagcaccgaggccgagacaatcctggacaacatcacccagagcacccagtccttcaacgacttcaccagagtcgtcggcggcgaggatgctaagcctggacagtttccttggcaagtggtgctgaacggcaaggtggacgctttttgtggcggctccatcgtgaacgagaagtggatcgtgaccgccgctcactgtgtggaaaccggcgtgaagattacagtggtggccggggagcacaacatcgaggaaacagagcacaccgagcagaaacggaacgtgatcagaatcatccctcaccacaactacaacgccgccatcaacaagtacaaccacgacattgccctgctcgagctggacgaacccctggtcctgaactcttacgtgacccctatctgtatcgccgacaaagagtacaccaacatctttctgaagttcggcagcggctacgtgtccggctggggaagagttttccacaagggcagatcagccctggtgctgcagtacctgagagtgcccctggtggatagagccacatgcctgctgagcaccaagttcaccatctacaacaacatgttctgcgccggcttccacgaaggcggcagagattcttgtcagggcgattctggcggccctcacgtgacagaagtcgagggcacatcttttctgaccggcatcatcagctggggcgaagagtgtgccatgaaggggaagtacggcatctataccaaggtgtccagatacgtgaactggatcaaagaaaagaccaagctcaccggatccggagaaggacggggaagcctgcttacatgcggagatgtggaggagaatcctggtcccatggtctttaccctggaagatttcgtcggcgactggcggcagacagccggctataatctggaccaggtgctggaacaaggcggagtgtccagcctgttccagaatctgggagtgtccgtgacacccatccagcggattgtgctgtctggcgagaacggcctgaagatcgacatccacgtgatcatcccttacgagggcctgagcggcgatcagatgggacagatcgagaagattttcaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacgggaacaagatcatcgacgagcggctgatcaaccccgatggcagcctgctgttcagagtgaccattaacggcgtgacaggctggcggctgtgcgaaaggattctggcctgatgatctagagatctcatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcctcgagaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 42 - DNA, 4651 bp, Artificial Sequence
  Synthetic Sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatggcgcgcctccagatatcatacactcgagcaactttgtatagaaaagttggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgcaagaattcacgcgtcaattgctcgaggccaccatggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctgatgagatgcattcgaagcggccgcgagctcaagcttgcaattccgataacttgtttattgcagcttataatggttacaaataaagcaatagcatcacaaatttcacaaataaagcatttttttcactgcattctagttgtggtttgtccaaactcatcaatgtatcttatcatgtctggcgctagcagcacaagtttgtacaaaaaagcaggctcctcgcaggaggcctggccggcctccggattacttccggtgcaggcgtcagctgcatcttgctggccagctcggaggactgtccccgtaactcccgtccgctgtgtatcaacacagctctagccaaattgttgaccgaacgacggggttcgattgtatcattcctcacatccaacacttgagtagtacaatgccttgtctaagtgcgcttgggacccagtctattttgtttgtgtgcacaagacgatactcctcgatcacgtaccaagcgcaggctggccctaacgacgtggtattctcgtattggtgtgccccagattgcataaggcactcatcacctgcgtttatgacgctggacgcctgcgtatgttgcaggactcctatgatgtcgctagcctgatgcgttgttatgtgattgaggtttagtacttggcaatatcattagatggaactaatacgcggtctaatcgttacaggtttccgacgattgagaagtatcacctgattctcgggagcgacttgtaactctactacacttattgatttggtactcctatgtgttgctatcttgactccgtaaatcgccgtgatacactctcttcgtttctcggcaatctatggctctaaactatacacaggcattctaagaataatctgacctgtccctgtagtactggtaggttactaccagtacggcgacaatgtgggagctacggcgtggtagaaccgggtacagacacagtccctgatcccgatagtaacactgtgcccgtagcagtgataatcattgcgcctcaacctgaggaagataagggaggcagtatatcgtgctattccctctattctcccacatgcgtatggcctgaggtcaatgatcaggtacatgccgtaatatgaggttttacgtagccgatcacgaaaaggttgctaaatacttctcgtcatatcccagctcattgttgggattatcgctaaacaggctaagttctaatgcccgtgggcatggcctcacgatctatggtgaaacagacagcccgatttagcaatgctacagagcacagtggttaatattggttgatacgtagaaccatgatcactgctcaattcgtgatgaagttgttcaatatacctagaagccactcaaggattctctctgtgtcactaatagaggtggtagagtgtttggaagccggttccacacagatttagagtgtcacctaattgccgtttatctaaggaattacgacaggagattccgaagtaattaggctattgcagagatacaggctaggtgaggtggagggtgtcttgcggtcttcgtccagccgccttgaaagcggggtttttttcaagcatcgctgcgtcattgcgtggtcgagacttatgcgccctacgaagttcgtctgaagtagtccaggaaagacctactttgcagttatcttcgcattcccacactcaccactacaactactcttccctcaatttcccggttagtttcgctaagctccgaccttgggttactgtgttgcatccgactcgctgcggctttctagtacgctgtactgtttcattcttctgtaggtctggttccgtaagtccgaatttccaggccgtggtctagtcctaattattttctgtcccggtagctatatttagccgagggtttgtccatttgcccggcgtagagcgccgcgtttgcgaacatttgcgcccgtaatacgtagggacaccgtcgggtaatggatggcaaaagccgaaaacggcgtcttccggcgcttggattcagcgctcttgagccataaaccgcgttgcttctttggttaattcgtattaatgatcctaagcgccagcttattcgttaagaggcactaggcgcgccgcggcatgcgatcgccagcatggctacgacccagctttcttgtacaaagtggtgatggccggccgcttcgagttaattaatccaaccggttaccgcctaggatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 43 - DNA, 7302 bp, Artificial Sequence
  Synthetic Sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggagatcggtacttcgcgaatgcgtcgagatggtaccaccggtactgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcttaagcctaggcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggatatccgaggacagggccctgtctcctcagcttcaggcaccaccactgacctgggacagtgaatcgcaagaattcacgcgtcaattgctcgaggccaccatggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctgatgagatgcattcgaagcggccgcgagctcaagcttgcaattccgataacttgtttattgcagcttataatggttacaaataaagcaatagcatcacaaatttcacaaataaagcatttttttcactgcattctagttgtggtttgtccaaactcatcaatgtatcttatcatgtctggcgctagcagcacctcgcaggaggcctggccggcctccggattacttccggtgcaggcgtcagctgcatcttgctggccagctcggaggactgtccccgtaactcccgtccgctgtgtatcaacacagctctagccaaattgttgaccgaacgacggggttcgattgtttcattcctcacatccaacacttgagtagtacaatgccttgtctaagtgcgcttgggacccagtctattttgtttgtgtgcacaagacgatactcctcgatcacgcaccaagcgcaggctggccctaacgacgtggtattctcgtattggagagccccagattgcataaggcactcatcacctgcgtttatgacgctggacgcctgcgtatgttgcaggactcctatgatgtcgcttgcctgatgcgttgttatgtgattgaggtttagtacttggcaatatcattagatggaactaatacgcggtctaatcgttacaggtttccgacgattgagaagtatcacctgattctcgggagcgacttgtaacactactacacttattgatttggtactcctatgtgttgctatcttgactccgtaaatcgccgtgatacactctcttcgtttctcggcaatctatggctctaaactatacacaggcattctaagaataatctgacctgtccctgtagtactggtaggttactaccagtacggcgacaatgtgggagctacggcgtggtagaaccgggtacagacacagtccctgatcccgatagtaacactgtgcccgtagcagtgataatcattgcgcctcaacctgaggaagataagggaggcagtatatcgtgctattccctctattctcccacatgcgtatggcctgaggtcaatgttcaggtacatgccgtaatatgaggttttacgtagccgatcacgaaaaggttgctaaatacttctcgtcatatcccagctcattgttgggattatcgctaaacaggctaagttctaatgcccgtgggcatggcctcacgatctatggtgatacagacagcccgatttagcaatgcttcagagcacagtggtaaatattggttgatacgtagaaccatgatcactgctcaattcgtgatgaagttgttcaatatacctagaagccactcaaggattctctctgagtcactaatagaggtggtagagtgtttggaagccggttccacacagatttagagtgtcacctaattgccgtttatctaaggaattacgacaggagataccgaagtaattaggctattgcagagatacaggctaggtgaggtggagggtgtcttgcggtcttcgtccagccgccttgaaagcggggtttttttcaagcatcgctgcgtcattgcgtggtcgagacttatgcgccctacgaagttcgtctgaagtagtccaggaaagacctactttgcagttatcttcgcattcccacactcaccactacaactactcttccctcaatttcccggttagtttcgctaagctccgaccttgggttactgtgttgcatccgactcgctgcggctttctagtacgctgtactgtttcattcttctgtaggtctggttccgtaagtccgaatttccaggccgtggtctagtcctaattattttctgtcccggtagctatatttagccgagggtttgtccatttgcccggcgtagagcgccgcgtttgcgaacatttgcgcccgtaatacgtagggacaccgtcgggtaatggatggcaaaagccgaaaacggcgtcttccggcgcttggattcagcgctcttgagccataaaccgcgttgcttctttggttaattcgtattaatgatcctaagcgccagcttattcgttaagaggcactaggcgcgccgcggcatgcgatcgccagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagactagtgtcgacatcggatgccgggaccgacgagtgcagaggcgtgcaagcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 44 - protein, 594 aa, Artificial Sequence
  Synthetic Sequence
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 45 - protein, 594 aa, Artificial Sequence
  Synthetic Sequence
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 46 - protein, 593 aa, Artificial Sequence
  Synthetic Sequence
GSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 47 - protein, 593 aa, Artificial Sequence
  Synthetic Sequence
GSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 48 - protein, 7 aa, Artificial Sequence
  Synthetic Sequence
PKKKRKV

SEQ ID NO: 49 - DNA, 21 bp, Artificial Sequence
  Synthetic Sequence
cccaagaagaagcggaaagtt

SEQ ID NO: 50 - protein, 5 aa, Artificial Sequence
  Synthetic Sequence
GGGGS

SEQ ID NO: 51 - DNA, 15 bp, Artificial Sequence
  Synthetic Sequence
ggcggcggaggcagc

SEQ ID NO: 52 - protein, 605 aa, Artificial Sequence
  Synthetic Sequence
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 53 - DNA, 1815 bp, Artificial Sequence
  Synthetic Sequence
atggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttc

SEQ ID NO: 54 - protein, 603 aa, Artificial Sequence
  Synthetic Sequence
PKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 55 - DNA, 1809 bp, Artificial Sequence
  Synthetic Sequence
cccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttc

SEQ ID NO: 56 - protein, 340 aa, Artificial Sequence
  Synthetic Sequence
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 57 - protein, 339 aa, Artificial Sequence
  Synthetic Sequence
GKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 58 - protein, 340 aa, Artificial Sequence
  Synthetic Sequence
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 59 - protein, 339 aa, Artificial Sequence
  Synthetic Sequence
GKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 60 - protein, 1496 aa, Artificial Sequence
  Synthetic Sequence
MSKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 61 - protein, 1495 aa, Artificial Sequence
  Synthetic Sequence
SKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 62 - protein, 649 aa, Artificial Sequence
  Synthetic Sequence
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 63 - protein, 648 aa, Artificial Sequence
  Synthetic Sequence
EEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 64 - protein, 636 aa, Artificial Sequence
  Synthetic Sequence
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 65 - protein, 635 aa, Artificial Sequence
  Synthetic Sequence
MLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 66 - DNA, 1866 bp, Artificial Sequence
  Synthetic Sequence
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 67 - DNA, 2214 bp, Artificial Sequence
  Synthetic Sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttccgggttacaaatacctcggacccggcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaacctgttaagacggctccgggaaaaaagaggccggtagagcactctcctgtggagccagactcctcctcgggaaccggaaaggcgggccagcagcctgcgaggaagcgactcaactttggtcagactggagacaccgactccgccgctgacccccagcctctcggagaaccaccagcagccccctctggtctgggaactggtacaatggctgcaggcggtggcgctccaatggcagacaataacgaaggcgccgacggagtgggtaatgcctcgggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtactttgacttcaaccgcttccactgccacttctcaccacgtgactggcaaagactcatcaacaacaactggggattccggcccaagagactcaacttcaagctcttcaacatccaggtcaaggaggtcacgcagaatgaaggcaccaagaccatcgccaataaccttaccagcacggttcaggtgtttactgactcggagtaccagctgccgtacgttctcggctctgcccaccagggctgcctgcctccgttcccggcggacgtgttcatgattccgcagtacggctacctaacgctcaacaatggcagccaggcgatgggtcgctcgtccttctactgcctggagtactttccgtcgcagatgctgagaaccggcaacaacttccagtttacttacaccttcgaggacgtgcctttccacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggcggctttggcctgaaacatcctccgcctcagatcctgatcaagaacacgcctgtacctgcggatcctccaacggccttcaacaaggacaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 68 - DNA, 5336 bp, Artificial Sequence
  Synthetic Sequence
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcg

SEQ ID NO: 69 - DNA, 3944 bp, Artificial Sequence
  Synthetic Sequence
cccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacg

SEQ ID NO: 70 - DNA, 1866 bp, Artificial Sequence
  Synthetic Sequence
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 71 - DNA, 2208 bp, Artificial Sequence
  Synthetic Sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggcaagacaggccagcagcccgctaaaaagagactcaattttggtcagactggcgactcagagtcagtcccagaccctcaacctctcggagaaccaccagcagccccctctggtctgggaactaatacgatggctacaggcagtggcgcaccaatggcagacaataacgagggtgccgatggagtgggtaattcctcaggaaattggcattgcgattcccaatggctgggcgacagagtcatcaccaccagcacccgaacctgggccctgcccacctacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtgggctggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcgacacacaaggcgttcttccaggcatggtatggcaggacagagatgtgtaccttcagggacccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactcaacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 72 - DNA, 16 bp, Artificial Sequence
  Synthetic Sequence
atgatttaaatcaggt

SEQ ID NO: 73 - DNA, 141 bp, Artificial Sequence
  Synthetic Sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 74 - DNA, 255 bp, Artificial Sequence
  Synthetic Sequence
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgca

SEQ ID NO: 75 - DNA, 122 bp, Artificial Sequence
  Synthetic Sequence
aacttgtttattgcagcttataatggttacaaataaagcaatagcatcacaaatttcacaaataaagcatttttttcactgcattctagttgtggtttgtccaaactcatcaatgtatctta

SEQ ID NO: 76 - DNA, 1906 bp, Artificial Sequence
  Synthetic Sequence
cctcgcaggaggcctggccggcctccggattacttccggtgcaggcgtcagctgcatcttgctggccagctcggaggactgtccccgtaactcccgtccgctgtgtatcaacacagctctagccaaattgttgaccgaacgacggggttcgattgtatcattcctcacatccaacacttgagtagtacaatgccttgtctaagtgcgcttgggacccagtctattttgtttgtgtgcacaagacgatactcctcgatcacgtaccaagcgcaggctggccctaacgacgtggtattctcgtattggtgtgccccagattgcataaggcactcatcacctgcgtttatgacgctggacgcctgcgtatgttgcaggactcctatgatgtcgctagcctgatgcgttgttatgtgattgaggtttagtacttggcaatatcattagatggaactaatacgcggtctaatcgttacaggtttccgacgattgagaagtatcacctgattctcgggagcgacttgtaactctactacacttattgatttggtactcctatgtgttgctatcttgactccgtaaatcgccgtgatacactctcttcgtttctcggcaatctatggctctaaactatacacaggcattctaagaataatctgacctgtccctgtagtactggtaggttactaccagtacggcgacaatgtgggagctacggcgtggtagaaccgggtacagacacagtccctgatcccgatagtaacactgtgcccgtagcagtgataatcattgcgcctcaacctgaggaagataagggaggcagtatatcgtgctattccctctattctcccacatgcgtatggcctgaggtcaatgatcaggtacatgccgtaatatgaggttttacgtagccgatcacgaaaaggttgctaaatacttctcgtcatatcccagctcattgttgggattatcgctaaacaggctaagttctaatgcccgtgggcatggcctcacgatctatggtgaaacagacagcccgatttagcaatgctacagagcacagtggttaatattggttgatacgtagaaccatgatcactgctcaattcgtgatgaagttgttcaatatacctagaagccactcaaggattctctctgtgtcactaatagaggtggtagagtgtttggaagccggttccacacagatttagagtgtcacctaattgccgtttatctaaggaattacgacaggagattccgaagtaattaggctattgcagagatacaggctaggtgaggtggagggtgtcttgcggtcttcgtccagccgccttgaaagcggggtttttttcaagcatcgctgcgtcattgcgtggtcgagacttatgcgccctacgaagttcgtctgaagtagtccaggaaagacctactttgcagttatcttcgcattcccacactcaccactacaactactcttccctcaatttcccggttagtttcgctaagctccgaccttgggttactgtgttgcatccgactcgctgcggctttctagtacgctgtactgtttcattcttctgtaggtctggttccgtaagtccgaatttccaggccgtggtctagtcctaattattttctgtcccggtagctatatttagccgagggtttgtccatttgcccggcgtagagcgccgcgtttgcgaacatttgcgcccgtaatacgtagggacaccgtcgggtaatggatggcaaaagccgaaaacggcgtcttccggcgcttggattcagcgctcttgagccataaaccgcgttgcttctttggttaattcgtattaatgatcctaagcgccagcttattcgttaagaggcactaggcgcgccgcggcatgcgatcgccagcatggctacg

SEQ ID NO: 77 - DNA, 141 bp, Artificial Sequence; identical to SEQ ID NO: 18
  Synthetic Sequence

SEQ ID NO: 78 - DNA, 4651 bp, Artificial Sequence; identical to SEQ ID NO: 42
  Synthetic Sequence

SEQ ID NO: 79 - DNA, 238 bp, Artificial Sequence
  Synthetic Sequence
ccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 80 - DNA, 309 bp, Artificial Sequence; identical to SEQ ID NO: 21
  Synthetic Sequence